(19) United States
(12) Patent Application Publication
(10) Pub. No.: US 2022/0380815 A1
(43) Pub. Date: Dec. 1, 2022

(54) PRODUCTION OF MALONATE SEMIALDEHYDE AND DERIVATIVES BY MICROORGANISMS EXPRESSING ASPARTATE 1-DECARBOXYLASE

(71) Applicant: Braskem S.A., Camacari (BR)

(72) Inventors: Felipe GALZERANI, Campinas (BR); Bianca Bassetto BISSONI, Campinas (BR); Karine JAILLARDON, Saint-Michel-sur-Orge (FR); Dominique LOUIS, Forges Les Bains (FR)

(21) Appl. No.: 17/830,229

(22) Filed: Jun. 1, 2022

Related U.S. Application Data
(60) Provisional application No. 63/195,447, filed on Jun. 1, 2021.

(51) Int. Cl. C12P 7/40; C12N 15/52; C12N 1/18; C12N 15/81

(52) U.S. Cl. CPC C12P 7/40; C12N 15/52; C12Y 401/01011; C12Y 206/01001; C12Y 206/01019; C12N 1/18; C12N 15/81; C12N 2800/102

(57) ABSTRACT

The present disclosure provides recombinant microorganisms and methods for producing malonate semialdehyde and/or related products, such as ketones, alcohols, organic acids, esters, alkenes, amino acids, and combinations thereof including 3-hydroxypropionic acid, acrylic acid, propionic acid, 1-propanol, acetone, 2-propanol, butanone, 1-butanol, 2-butanol, methyl propionate, 1,3-propanediol, isoamyl alcohol, 1,3-butanediol, 1,4-butanediol, 2,3-butanediol, lactic acid, adipic acid, glutamic acid, itaconic acid, ethyl acetate, isopropyl acetate, acetic acid, butyric acid, caproic acid, citric acid, methacrylic acid, succinic acid, propylene, butadiene, ethanol, isoprenol, leucine, isoleucine, glutamine, glycine, and isoprene, from β-alanine. The recombinant microorganism expresses an asparate 1-decarboxylase that catalyzes the production of malonate semialdehyde from β-alanine.

### CROSS-REFERENCE TO RELATED APPLICATION

This U.S. Patent application claims benefit of U.S. Provisional Patent Application No. 63/195,447, filed on Jun. 1, 2021, which is incorporated herein by reference in its entirety.

### BACKGROUND

Malonate semialdehyde (MSA) and its derivatives can be used to produce several products of industrial interest that typically are obtained through petrochemical extraction processes, which are generally considered to have a detrimental effect on the environment. In contrast, bioprocesses for producing chemicals of industrial importance from renewable materials are regarded as significantly more environmentally friendly. However, bioprocesses for the production industrially relevant chemicals frequently suffer from poor efficiency and low product yields.

There is therefore a need for more efficient and higher-yielding bioprocesses for producing MSA and its derivative chemicals of commercial importance. Relatedly, there is a need for more efficient enzymes so as to increase pathway yields and productivity, and make bio-based technologies, processes, and products more cost-competitive against their petro-based counterparts.

### SUMMARY

This disclosure provides a recombinant microorganism comprising: (a) at least one nucleic acid molecule encoding an aspartate 1-decarboxylase that catalyzes the production of β-alanine from aspartate; and (b) at least one nucleic acid molecule encoding one or more polypeptides that catalyze the production of malonate semialdehyde (MSA) from β-alanine. The aspartate 1-decarboxylase is of the Class Malacostraca, Entognatha, Amphibia, Aves, or Actinistia, including aspartate 1-decarboxylases derived from the Class Malacostraca, Entognatha, Amphibia, Aves, or Actinistia, and/or variants thereof.

In some embodiments, the aspartate 1-decarboxylase is of the Class Malacostraca or Entognatha, wherein the aspartate 1-decarboxylase comprises: (a) a glutamine at a residue corresponding to position 333 of the amino acid sequence of SEQ ID NO: 1, and a partial amino acid sequence having at least 75% sequence identity to amino acids 338-473 of SEQ ID NO: 1; (b) a glutamine at a residue corresponding to position 378 of the amino acid sequence of SEQ ID NO: 2, and a partial amino acid sequence having at least 75% sequence identity to amino acids 383-519 of SEQ ID NO: 2; (c) a glutamine at a residue corresponding to position 340 of the amino acid sequence of SEQ ID NO: 3, and a partial amino acid sequence having at least 75% sequence identity to amino acids 345-483 of SEQ ID NO: 3; (d) a glutamine at a residue corresponding to position 320 of the amino acid sequence of SEQ ID NO: 4, and a partial amino acid sequence having at least 75% sequence identity to amino acids 325-457 of SEQ ID NO: 4; (e) a glutamine at a residue corresponding to position 353 of the amino acid sequence of SEQ ID NO: 5, and a partial amino acid sequence having at least 75% sequence identity to amino acids 358-490 of SEQ ID NO: 5; (f) a glutamine at a residue corresponding to position 320 of the amino acid sequence of SEQ ID NO: 6, and a partial amino acid sequence having at least 75% sequence identity to amino acids 325-457 of SEQ ID NO: 6; (g) a glutamine at a residue corresponding to position 335 of the amino acid sequence of SEQ ID NO: 7, and a partial amino acid sequence having at least 75% sequence identity to amino acids 340-472 of SEQ ID NO: 7; (h) a glutamine at a residue corresponding to position 312 of the amino acid sequence of SEQ ID NO: 8, and a partial amino acid sequence having at least 75% sequence identity to amino acids 317-453 of SEQ ID NO: 8; (i) a glutamine at a residue corresponding to position 310 of the amino acid sequence of SEQ ID NO: 9, and a partial amino acid sequence having at least 75% sequence identity to amino acids 315-459 of SEQ ID NO: 9; or (j) a glutamine at a residue corresponding to position 380 of the amino acid sequence of SEQ ID NO: 10, and a partial amino acid sequence having at least 75% sequence identity to amino acids 385-505 of SEQ ID NO: 10.

In some embodiments, the aspartate 1-decarboxylase is of the Class Amphibia, Aves, or Actinistia, wherein the aspartate 1-decarboxylase comprises: (a) an isoleucine at a residue corresponding to position 320 of the amino acid sequence of SEQ ID NO: 11, and a partial amino acid sequence having at least 75% sequence identity to amino acids 325-458 of SEQ ID NO: 11; (b) an isoleucine at a residue corresponding to position 337 of the amino acid sequence of SEQ ID NO: 12, and a partial amino acid sequence having at least 75% sequence identity to amino acids 342-475 of SEQ ID NO: 12; (c) an isoleucine at a residue corresponding to position 329 of the amino acid sequence of SEQ ID NO: 13, and a partial amino acid sequence having at least 75% sequence identity to amino acids 334-467 of SEQ ID NO: 13; (d) an isoleucine at a residue corresponding to position 328 of the amino acid sequence of SEQ ID NO: 14, and a partial amino acid sequence having at least 75% sequence identity to amino acids 333-466 of SEQ ID NO: 14; (e) an isoleucine at a residue corresponding to position 318 of the amino acid sequence of SEQ ID NO: 15, and a partial amino acid sequence having at least 75% sequence identity to amino acids 322-455 of SEQ ID NO: 15; (f) an isoleucine at a residue corresponding to position 319 of the amino acid sequence of SEQ ID NO: 16, and a partial amino acid sequence having at least 75% sequence identity to amino acids 323-457 of SEQ ID NO: 16; (g) an isoleucine at a residue corresponding to position 329 of the amino acid sequence of SEQ ID NO: 17, and a partial amino acid sequence having at least 75% sequence identity to amino acids 334-467 of SEQ ID NO: 17; (h) an isoleucine at a residue corresponding to position 329 of the amino acid sequence of SEQ ID NO: 18, and a partial amino acid sequence having at least 75% sequence identity to amino acids 334-467 of SEQ ID NO: 18; (i) an isoleucine at a residue corresponding to position 329 of the amino acid sequence of SEQ ID NO: 19, and a partial amino acid sequence having at least 75% sequence identity to amino acids 334-467 of SEQ ID NO: 19; or (j) an isoleucine at a residue corresponding to position 317 of the amino acid sequence of SEQ ID NO: 20, and a partial amino acid sequence having at least 75% sequence identity to amino acids 322-455 of SEQ ID NO: 20.

In an embodiment, the aspartate 1-decarboxylase is from Folsomia candida, Orchesella cincta, Paralithodes camtschaticus, Neocaridina davidi, Cheraz quadricarinatus, Stenopus hispidus, Panulirus ornatus, Birgus latro, Scylla olivacea, or Litopenaeus vannamei.

In an embodiment, the aspartate 1-decarboxylase is from Egretta garzetta, Latimeria chalumnae, Coturnix japonica, Serinus canaria, Xenopus tropicalis, Nipponia nippon, Xenopus tropicalis, Daphnia magna, Phasianus colchicus, or Xenopus laevis.

In an embodiment, the polypeptide that catalyzes the production of MSA from (3-alanine is a β-alanine pyruvate amino transferase and/or a β-alanine transaminase, preferably wherein the β-alanine pyruvate amino transferase and/or a β-alanine transaminase is classified as EC number 2.6.1.-, EC number 2.6.1.19, and/or EC number 2.6.1.18.

In an embodiment, the recombinant microorganism further comprises at least one nucleic acid molecule encoding one or more polypeptides that catalyze the production of 3-hydroxypropionic acid (3-TIP) from malonate semialdehyde.

In an embodiment, the polypeptide that catalyzes the production of 3-HP from MSA is a 3-hydroxypropionic acid dehydrogenase, preferably wherein the 3-hydroxypropionic acid dehydrogenase is classified as EC number 1.1.1.-, EC number 1.1.1.298, and/or EC number 1.1.1.59.

In an embodiment, the recombinant microorganism further comprises at least one nucleic acid molecule encoding one or more polypeptides that catalyze the production of a derivative selected from 1-propanol, propionic acid, acrylic acid, butanone, 2-butanol, methyl propionate, succinic acid, 1,4-butanediol, propylene, or a combination thereof from 3-HP.

In an embodiment, the microorganism is capable of producing 1-propanol, the recombinant microorganism further comprising: (a) at least one nucleic acid molecule encoding one or more polypeptides that catalyze the production of 3-HP-CoA from 3-HP; (b) at least one nucleic acid molecule encoding one or more polypeptides that catalyze the production of acrylyl-CoA from 3-HP-CoA; (c) at least one nucleic acid molecule encoding one or more polypeptides that catalyze the production of propionyl-CoA from acrylyl-CoA; (d) at least one nucleic acid molecule encoding one or more polypeptides that catalyze the production of propionaldehyde from propionyl-CoA; and (e) at least one nucleic acid molecule encoding one or more polypeptides that catalyze the production of 1-propanol from propionaldehyde.

In an embodiment, the microorganism comprises at least one nucleic acid molecule encoding: (a) a 3-hydroxypropionyl-CoA synthetase and/or a 3-hydroxypropionyl-CoA transferase, preferably wherein the 3-hydroxypropionyl-CoA synthetase and/or 3-hydroxypropionyl-CoA transferase is classified as EC number 2.8.3.1, EC number 6.2.1.17, and/or EC number 6.2.1.36; (b) a 3-hydroxypropionyl-CoA dehydratase and/or an enoyl-CoA hydratase, preferably wherein the 3-hydroxypropionyl-CoA dehydratase and/or enoyl-CoA hydratase is classified as EC number 4.2.1.116, EC number 4.2.1.55, EC number 4.2.1.150, and/or EC number 4.2.1.17; (c), an acrylyl-CoA reductase, preferably wherein the acrylyl-CoA reductase is classified as EC number 1.3.1.84 and/or EC number 1.3.1.95; and/or (d) a bifunctional alcohol/aldehyde dehydrogenase, preferably wherein the bifunctional alcohol/aldehyde dehydrogenase is classified as EC number 1.2.1.10 and/or EC number 1.1.1.1; an aldehyde dehydrogenase, preferably wherein the aldehyde dehydrogenase is classified as EC number 1.2.1.10; and/or an alcohol dehydrogenase, preferably wherein the alcohol dehydrogenase is classified as EC number 1.1.1.1 and/or EC number 1.1.1.2.

In an embodiment, the recombinant microorganism further comprises: (a) at least one nucleic acid molecule encoding one or more polypeptides that catalyze the production of 3-HP-CoA from 3-HP; and (b) at least one nucleic acid molecule encoding one or more polypeptides that catalyze the production of acrylyl-CoA from 3-HP-CoA.

In an embodiment, the polypeptide that catalyzes the production of 3-HP-CoA from 3-HP is a 3-hydroxypropionyl-CoA synthetase and/or a 3-hydroxypropionyl-CoA transferase, preferably wherein the 3-hydroxypropionyl-CoA synthetase and/or 3-hydroxypropionyl-CoA transferase is classified as EC number 2.8.3.1, EC number 6.2.1.17, and/or EC number 6.2.1.36.

In an embodiment, the polypeptide that catalyzes the production of acrylyl-CoA from 3-HP-CoA is a 3-hydroxypropionyl-CoA dehydratase and/or an enoyl-CoA hydratase, preferably wherein the 3-hydroxypropionyl-CoA dehydratase and/or enoyl-CoA hydratase is classified as EC number 4.2.1.116, EC number 4.2.1.55, EC number 4.2.1.150, and/or EC number 4.2.1.17.

In an embodiment, the recombinant microorganism further comprises at least one nucleic acid molecule encoding one or more polypeptides that catalyze the production of acrylic acid and/or acrylate from acrylyl-CoA.

In an embodiment, the polypeptide that catalyzes the production of acrylic acid and/or acrylate from acrylyl-CoA is an acyl-CoA hydrolase and/or a thioesterase, preferably wherein the acyl-CoA hydrolase and/or thioesterase is classified as EC number 3.2.1.-.

In an embodiment, the recombinant microorganism further comprises at least one nucleic acid molecule encoding one or more polypeptides that catalyze the production of propionyl-CoA from acrylyl-CoA.

In an embodiment, the polypeptide that catalyzes the production of propionyl-CoA from acrylyl-CoA is an acrylyl-CoA reductase, preferably wherein the acrylyl-CoA reductase is classified as EC number 1.3.1.84 and/or EC number 1.3.1.95.

In an embodiment, the recombinant microorganism further comprises at least one nucleic acid molecule encoding one or more polypeptides that catalyze the production of propionic acid from propionyl-CoA.

In an embodiment, the polypeptide that catalyzes the production of propionic acid from propionyl-CoA is a propionate CoA transferase, preferably wherein the propionate CoA transferase is classified as EC number 2.8.3.1.

In an embodiment, the polypeptides that catalyze the production of propionic acid from propionyl-CoA are: (a) a phosphotransacetylase, preferably wherein the phosphotransacetylase is classified as EC number 2.3.1.-.; and (b) an acetate kinase, preferably wherein the acetate kinase is classified as EC number 2.7.2.1.

In an embodiment, the recombinant microorganism further comprises at least one nucleic acid molecule encoding one or more polypeptides that catalyze the production of 1-propanol from propionyl-CoA.

In an embodiment, the polypeptide that catalyzes the production of 1-propanol from propionyl-CoA is a bifunctional alcohol/aldehyde dehydrogenase, preferably wherein the bifunctional alcohol/aldehyde dehydrogenase is classified as EC number 1.2.1.10 and/or EC number 1.1.1.1; an aldehyde dehydrogenase, preferably wherein the aldehyde dehydrogenase is classified as EC number 1.2.1.10; and/or an alcohol dehydrogenase, preferably wherein the alcohol dehydrogenase is classified as EC number 1.1.1.1 and/or EC number 1.1.1.2.

In an embodiment, the recombinant microorganism further comprises at least one nucleic acid molecule encoding one or more polypeptides that catalyze the production of acetyl-CoA from MSA.

In an embodiment, the polypeptide that catalyzes the production of acetyl-CoA from MSA is a malonate semialdehyde dehydrogenase (acetylating), preferably wherein the malonate semialdehyde dehydrogenase (acetylating) is classified as EC number 1.2.1.18.

In an embodiment, the recombinant microorganism further comprises at least one nucleic acid molecule encoding one or more polypeptides that catalyze the production of a derivative selected from ketones, such as acetone and methyl ethyl ketone; alcohols, such as 2-propanol, 1-butanol, 2-butanol, 1,3-propanediol, isoamyl alcohol, 1,3-butanediol, 1,4-butanediol, 2,3-butanediol, and isoprenol; organic acids, such as acetic acid, butyric acid, lactic acid, adipic acid, glutamic acid, itaconic acid, caproic acid, citric acid, methacrylic acid and succinic acid; esters, such as ethyl acetate and isopropyl acetate; alkenes, such as propylene, butadiene and isoprene; amino acids, such as leucine, isoleucine, glutamine and glycine; or a combination thereof from acetyl-CoA.

In an embodiment, the recombinant microorganism further comprises at least one nucleic acid molecule encoding one or more polypeptides that catalyze the production of acetone from acetyl-CoA.

In an embodiment, the polypeptides that catalyze the production of acetone from acetyl-CoA are: (a) a thiolase, preferably wherein the thiolase is classified as EC number 2.3.1.9; (b) a CoA transferase, preferably wherein the CoA transferase is classified as EC number 2.8.3.8; and (c) a decarboxylase, preferably wherein the decarboxylase is classified as EC number 4.1.1.4.

In an embodiment, the recombinant microorganism further comprises at least one nucleic acid molecule encoding one or more polypeptides that catalyze the production of 2-propanol (isopropanol) from acetone.

In an embodiment, the polypeptide that catalyzes the production of 2-propanol from acetone is an isopropanol dehydrogenase, preferably wherein the isopropanol dehydrogenase is classified as EC number 1.1.1.80.

In an embodiment, the recombinant microorganism further comprises at least one nucleic acid molecule encoding one or more polypeptides that catalyze the production of methyl ethyl ketone from the condensation of acetyl-CoA and propionyl-CoA.

In an embodiment, the polypeptides that catalyze the production of methyl ethyl ketone from the condensation of acetyl-CoA and propionyl-CoA sequentially are: (a) a beta-ketothiolase, preferably wherein the beta-ketothiolase is classified as EC number 2.3.1.16; (b) a CoA transferase and/or a CoA hydrolase, preferably wherein the CoA transferase and/or a CoA hydrolase is classified as EC number 2.8.3.8; and (c) a decarboxylase, preferably wherein the decarboxylase is classified as EC number 4.1.1.4.

In an embodiment, the recombinant microorganism further comprises at least one nucleic acid molecule encoding one or more polypeptides that catalyze the production of propylene from 1-propanol and/or 2-propanol, wherein the polypeptide is an alcohol dehydratase, preferably wherein the alcohol dehydratase is classified as EC number 4.2.1.127.

In an embodiment, the aspartate 1-decarboxylase uses pyridoxal-5′-phosphate (PLP) as a cofactor.

In an embodiment, the aspartate 1-decarboxylase has at least 70% sequence identity to the amino acid sequence of SEQ ID NO: 1, 2, 3, 4, 5, 6, 7, 8, 9, 10, 11, 12, 13, 14, 15, 16, 17, 18, 19, or 20.

In an embodiment, the aspartate 1-decarboxylase has at least 80% sequence identity to the amino acid sequence of SEQ ID NO: 1, 2, 3, 4, 5, 6, 7, 8, 9, 10, 11, 12, 13, 14, 15, 16, 17, 18, 19, or 20.

In an embodiment, the aspartate 1-decarboxylase has at least 90% sequence identity to the amino acid sequence of SEQ ID NO: 1, 2, 3, 4, 5, 6, 7, 8, 9, 10, 11, 12, 13, 14, 15, 16, 17, 18, 19, or 20.

In an embodiment, the aspartate 1-decarboxylase has at least 95% sequence identity to the amino acid sequence of SEQ ID NO: 1, 2, 3, 4, 5, 6, 7, 8, 9, 10, 11, 12, 13, 14, 15, 16, 17, 18, 19, or 20.

In an embodiment, the aspartate 1-decarboxylase has 100% sequence identity to the amino acid sequence of SEQ ID NO: 1, 2, 3, 4, 5, 6, 7, 8, 9, 10, 11, 12, 13, 14, 15, 16, 17, 18, 19, or 20.

In an embodiment, the microorganism is selected from a bacterium, a fungus, or a yeast.

The disclosure additionally provides a method of producing MSA and derivatives obtained from an MSA intermediate. The method comprises contacting the recombinant microorganism of the disclosure with a fermentable carbon source under conditions sufficient to produce MSA and/or the derivatives.

In some embodiments, the recombinant microorganism further produces 3-HP, acrylic acid, propionic acid, 1-propanol, acetone, isopropanol (2-propanol), butanone, 1-butanol, 2-butanol, methyl propionate, 1,3-propanediol, isoamyl alcohol, 1,3-butanediol, 1,4-butanediol, 2,3-butanediol, lactic acid, adipic acid, glutamic acid, itaconic acid, ethyl acetate, isopropyl acetate, acetic acid, butyric acid, caproic acid, citric acid, methacrylic acid, succinic acid, propylene, butadiene, ethanol, isoprenol, leucine, isoleucine, glutamine, glycine, isoprene, or a combination thereof.

### BRIEF DESCRIPTION OF THE DRAWINGS

FIG. 1 depicts a pathway for production of malonate semialdehyde and its derivatives using an aspartate decarboxylase.

FIGS. 2A-2F depict an alignment generated using PatSnap Bio of SEQ ID NOs:1-20 and the aspartate 1-decarboxylase from Aedes aegytpi (UniProt ID Q171S0).

FIG. 3 is a schematic drawing of a phenoytype-based growth complementation screen to assess asparate decarboxylase activity.

FIGS. 4A-4B are images showing growth of transformants on SY-U and β-alanine as positive control and on YPDA as selective medium.

FIG. 5 is a schematic drawing of the CLU497 clusters for the expression of four different genes.

FIG. 6 is a schematic drawing of the reaction catalyzed by asparate decarboxylase and the results of a study assessing activity of recombinant yeast expressing various asparate decarboxylase enzymes.

### DETAILED DESCRIPTION

The present disclosure is directed to recombinant microorganisms that produce malonate semialdehyde and/or related products, such as ketones, alcohols, organic acids, esters, alkenes, amino acids, and combinations thereof including 3-hydroxypropionic acid, acrylic acid, propionic acid, 1-propanol, acetone, 2-propanol, butanone, 1-butanol, 2-butanol, methyl propionate, 1,3-propanediol, isoamyl alcohol, 1,3-butanediol, 1,4-butanediol, 2,3-butanediol, lactic acid, adipic acid, glutamic acid, itaconic acid, ethyl acetate, isopropyl acetate, acetic acid, butyric acid, caproic acid, citric acid, methacrylic acid, succinic acid, propylene, butadiene, ethanol, isoprenol, leucine, isoleucine, glutamine, glycine, and isoprene, from β-alanine, by expressing an asparate 1-decarboxylase. The present disclosure is also directed to methods of using recombinant microorganisms expressing an asparate 1-decarboxylase to produce malonate semialdehyde and/or related products, such as ketones, alcohols, organic acids, esters, alkenes, amino acids, and combinations thereof including 3-hydroxypropionic acid, acrylic acid, propionic acid, 1-propanol, acetone, 2-propanol, butanone, 1-butanol, 2-butanol, methyl propionate, 1,3-propanediol, isoamyl alcohol, 1,3-butanediol, 1,4-butanediol, 2,3-butanediol, lactic acid, adipic acid, glutamic acid, itaconic acid, ethyl acetate, isopropyl acetate, acetic acid, butyric acid, caproic acid, citric acid, methacrylic acid, succinic acid, propylene, butadiene, ethanol, isoprenol, leucine, isoleucine, glutamine, glycine, and isoprene.

As used herein, the singular forms “a,” “an,” and “the” include plural referents unless the context clearly dictates otherwise. Thus, for example, reference to “an enzyme” includes a plurality of such enzymes and reference to “the microorganism” includes reference to one or more microorganisms, and so forth.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.”

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, enzyme/protein “activity” and “function” are used interchangeably and designates, in the context of the disclosure, the capacity of (1) an enzyme to catalyze a desired reaction or (2) a protein to act in a certain manner.

As used herein, “aerobic conditions” refer to concentrations of oxygen in the culture medium that are sufficient for an aerobic or facultative anaerobic microorganism to use oxygen as a terminal electron acceptor.

As used herein, “anaerobic conditions” refer to culture or growth conditions with regard to the concentration of oxygen, which is intended to mean that the amount of oxygen is less than about 0% saturation of dissolved oxygen in liquid media. The term is also intended to include sealed chambers of liquid or solid media maintained with an atmosphere of less than about 0% oxygen.

As used herein, “microaerobic conditions” refer to concentrations of oxygen in the culture medium in which the concentration of oxygen is less than that in air under standard temperature and pressure, i.e., an oxygen concentration of up to ˜6% of the total gas present.

As used herein the terms “microorganism” or “microbe” should be taken broadly. These terms, used interchangeably, include but are not limited to, any organism that exists as a microscopic cell that is included within the domains of archaea, bacteria or eukarya, the latter including yeast and filamentous fungi, protozoa, algae, or higher Protista. Therefore, the term is intended to encompass prokaryotic or eukaryotic cells or organisms having a microscopic size and includes bacteria, archaea, and eubacteria of all species as well as eukaryotic microorganisms such as yeast and fungi. Also included are cell cultures of any species that can be cultured for the production of a chemical.

In some aspects, the disclosure provides a recombinant microorganism comprising: (a) at least one nucleic acid molecule encoding an aspartate 1-decarboxylase that catalyzes the production of β-alanine from aspartate; and (b) at least one nucleic acid molecule encoding one or more polypeptides that catalyze the production of malonate semialdehyde (MSA) from β-alanine. In some aspects, the aspartate 1-decarboxylase is of the Class Malacostraca, Entognatha, Amphibia, Aves, or Actinistia.

In some aspects, the microorganism is selected from a bacterium, a fungus, or a yeast. In some aspects, the recombinant microorganism is a yeast. In some aspects, the yeast is an ethanol-producing industrial yeast strain. In some aspects, the yeast is Saccharomyces cerevisiae. In some aspects, the yeast is capable of aerobic and anaerobic growth. In some aspects, the recombinant microorganism is derived from a parental microorganism selected from the group consisting of: Clostridium sp., Clostridium ljungdahlii, Clostridium autoethanogenum, Clostridium ragsdalei, Eubacterium limosum, Butyribacterium methylotrophicum, Moorella thermoacetica, Clostridium aceticum, Acetobacterium woodii, Alkalibaculum bacchii, Clostridium drakei, Clostridium carboxidivorans, Clostridium formicoaceticum, Clostridium scatologenes, Moorella thermoautotrophica, Acetonema longum, Blautia producta, Clostridium glycolicum, Clostridium magnum, Clostridium mayombei, Clostridium methoxybenzovorans, Clostridium acetobutylicum, Clostridium beijerinckii, Oxobacter pfennigii, Thermoanaerobacter kivui, Sporomusa ovata, Thermoacetogenium phaeum, Acetobacterium carbinolicum, Sporomusa termitida, Moorella glycerini, Eubacterium aggregans, Treponema azotonutricium, Escherichia coli, Saccharomyces cerevisiae, Pseudomonas putida, Bacillus sp., Candida sp., Candida Krusei, Corynebacterium sp., Yarrowia lipolytica, Scheffersomyces stipitis, and Terrisporobacter glycolicus.

In some aspects, the disclosure provides a method of producing MSA and/or derivatives comprising: contacting a recombinant microorganism as disclosed herein with a fermentable carbon source under conditions sufficient to produce MSA and/or derivatives. In some aspects, the recombinant microorganism produces a ketone, an alcohol, an organic acid, an ester, an alkene, an amino acid, or a combination thereof. In some aspects, the recombinant microorganism produces 3-HP, acrylic acid, propionic acid, 1-propanol, acetone, isopropanol (2-propanol), butanone, 1-butanol, 2-butanol, methyl propionate, 1,3-propanediol, isoamyl alcohol, 1,3-butanediol, 1,4-butanediol, 2,3-butanediol, lactic acid, adipic acid, glutamic acid, itaconic acid, ethyl acetate, isopropyl acetate, acetic acid, butyric acid, caproic acid, citric acid, methacrylic acid, succinic acid, propylene, butadiene, ethanol, isoprenol, leucine, isoleucine, glutamine, glycine, isoprene, or a combination thereof. In some aspects, the conditions comprise aerobic conditions. In some aspects, the conditions comprise microaerobic conditions. In some aspects, the conditions comprise anaerobic conditions.

### Production of Malonate Semialdehyde by Recombinant Microorganisms Expressing an Asparate 1-Decarboxylase

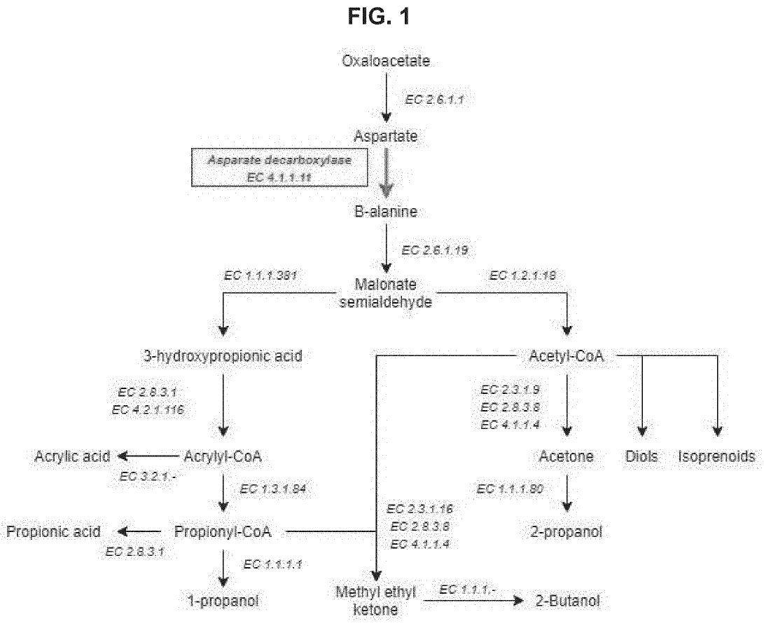

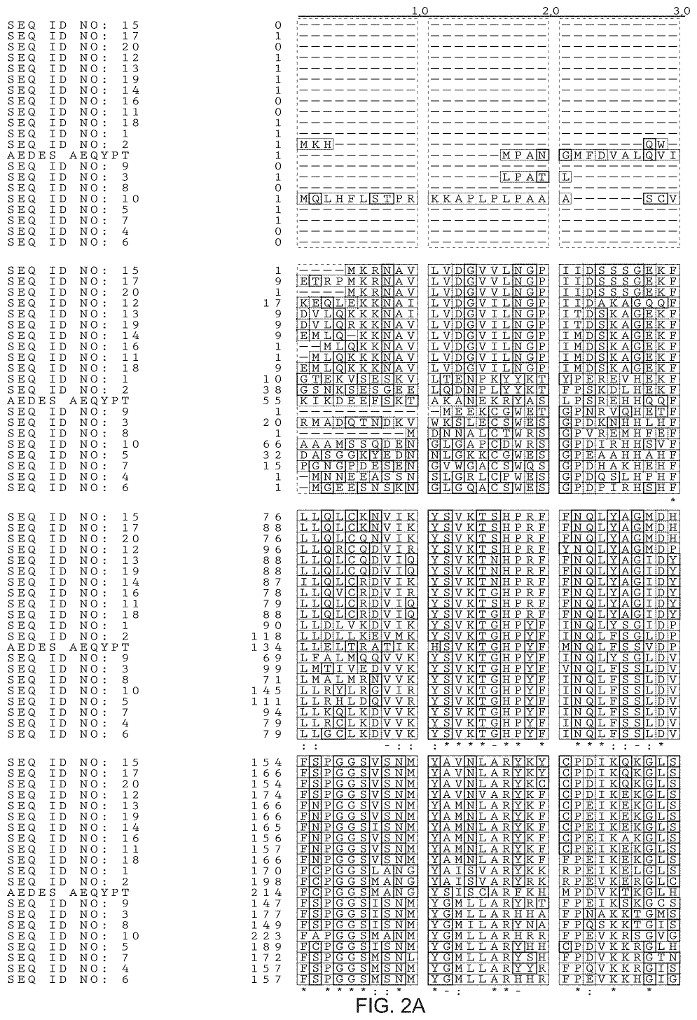

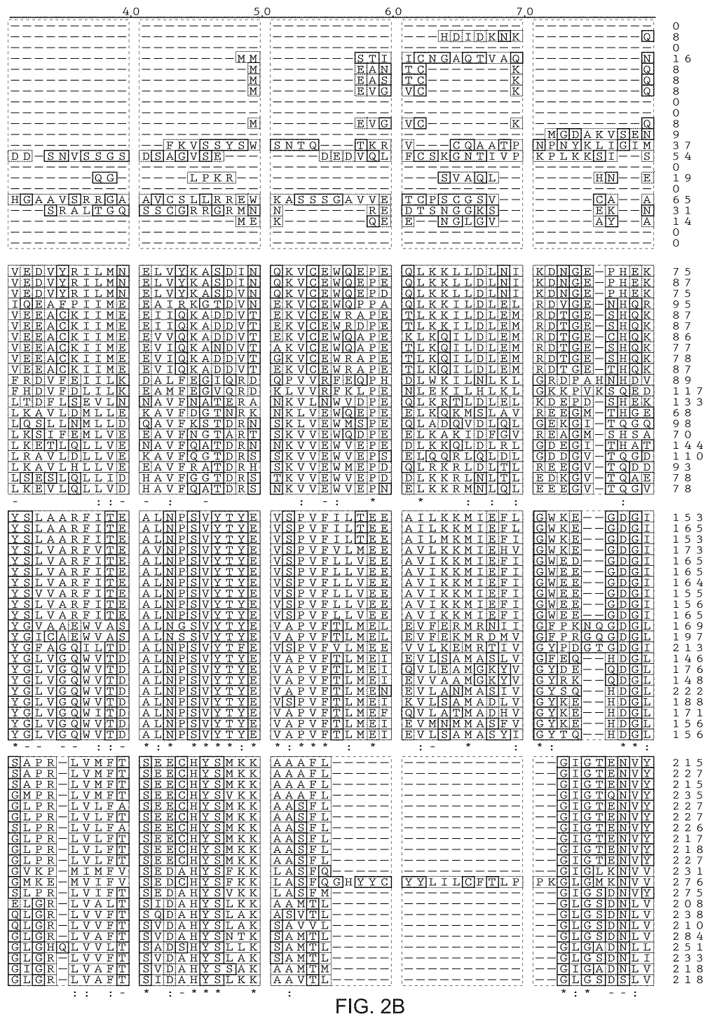

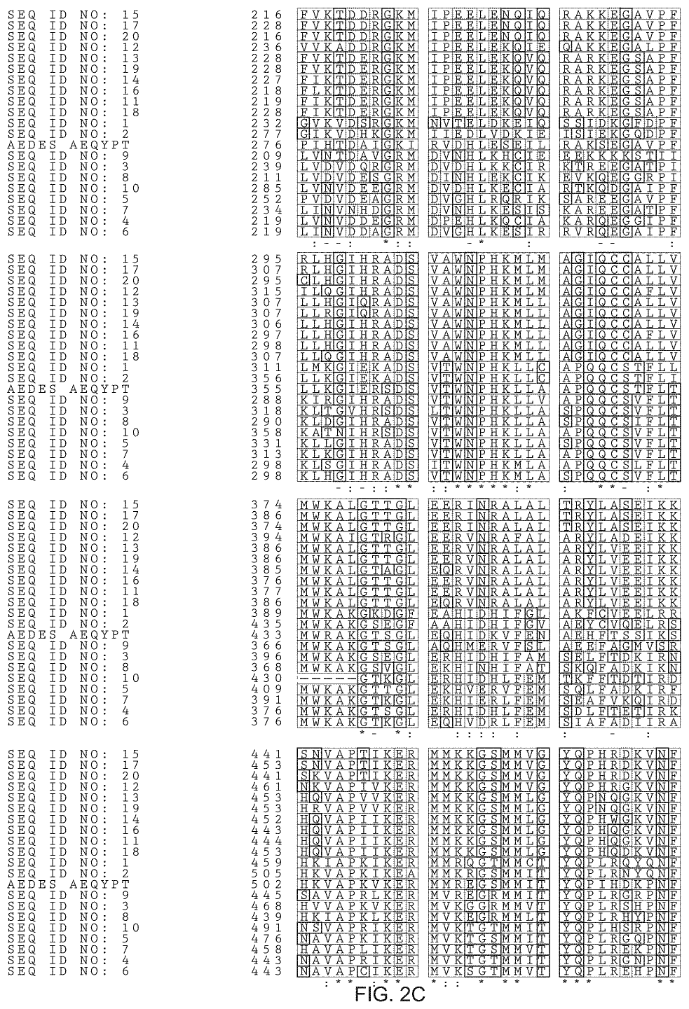

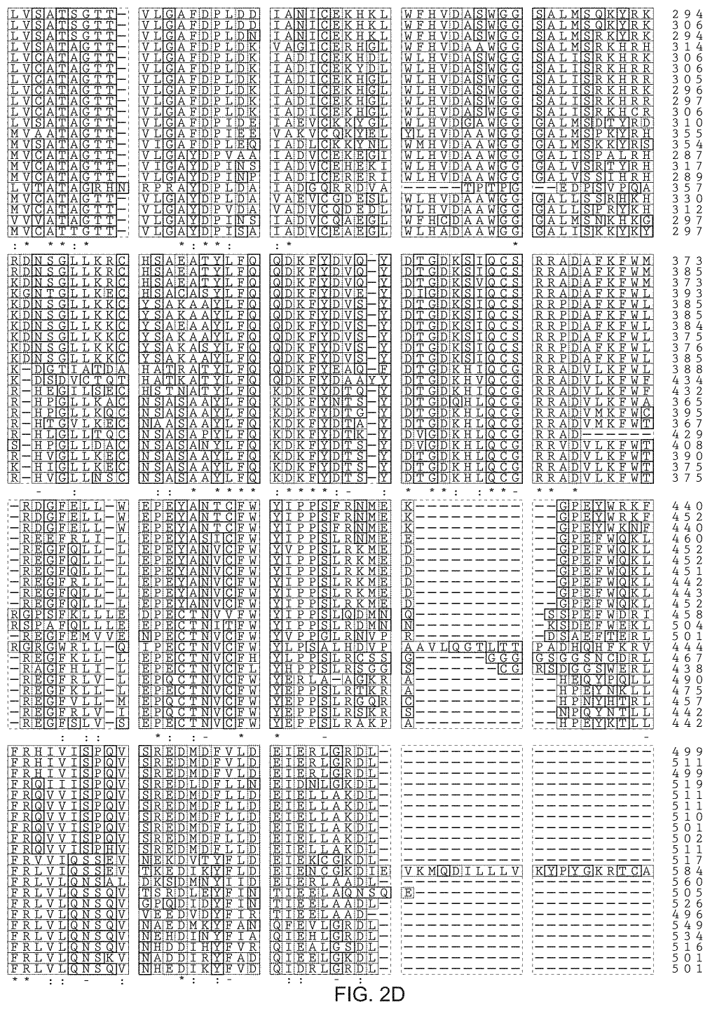

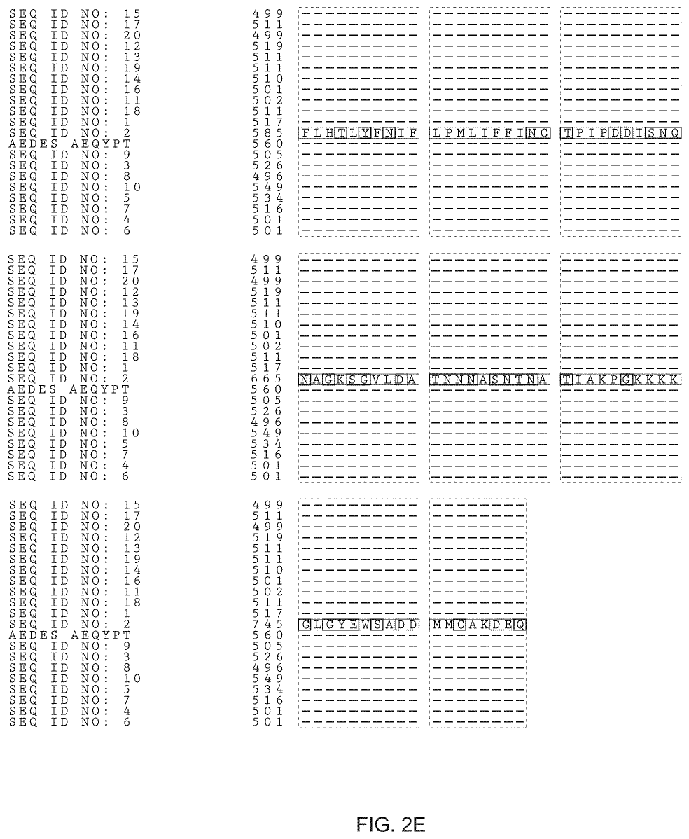

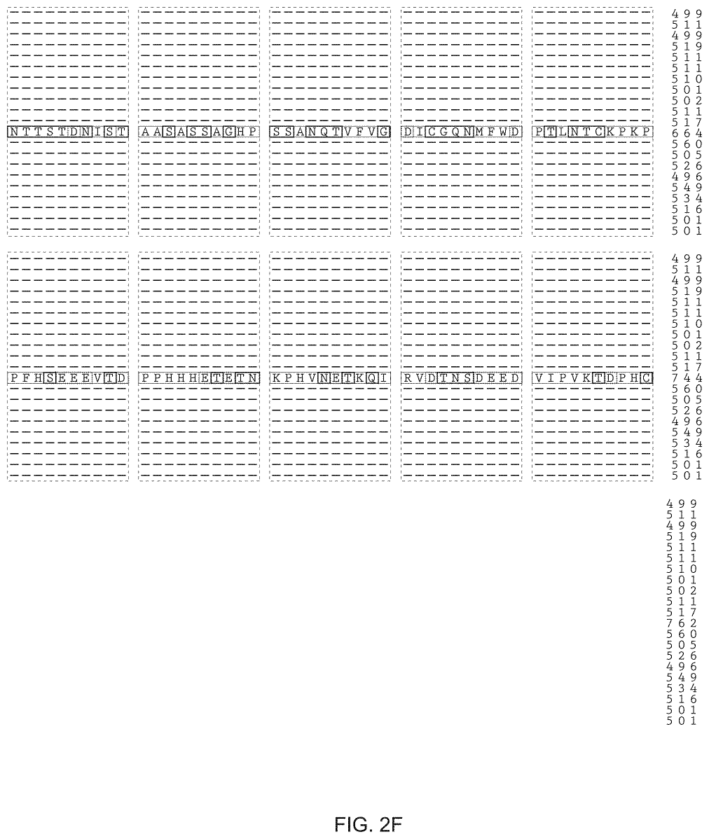

In some aspects, malonate semialdehyde and related products can be obtained from recombinant microorganisms expressing an asparate 1-decarboxylase by the steps shown in FIG. 1.

In some aspects, phosphoenolpyruvate (PEP) can be converted to oxaloacetate by a bacterial PEP carboxylase and/or PEP carboxykinase. In some aspects, the recombinant microorganism comprises one or more PEP carboxylases and/or PEP carboxykinases including, but not limited to, enzymes with EC number 4.1.1.31 and/or EC number 4.1.1.49. In some aspects, the PEP carboxylase (ppc) is from Escherichia coli. In some aspects, the PEP carboxykinase (pepck) is from Escherichia coli.

In some aspects, oxaloacetate can be converted to asparatate by one or more polypeptides that catalyze the production of aspartate from oxaloacetate, e.g., by amination of oxaloacetate by an aspartate aminotransferase. In some aspects, the recombinant microorganism comprises one or more aspartate aminotransferases including, but not limited to, enzymes with EC number 2.6.1.1. In some aspects, the aspartate aminotransferase (aat2) is from Sacchoromyces cerevisiae.

As shown in FIG. 1, β-alanine is obtained from aspartate by decarboxylation of aspartate via an aspartate 1-decarboxylase. In some aspects, the aspartate 1-decarboxylase is of the Class Malacostraca or Entognatha. In some aspects, the aspartate 1-decarboxylase is of the Class Amphibia, Aves, or Actinistia. In some aspects, the aspartate 1-decarboxylase is from Folsomia candida, Orchesella cincta, Paralithodes camtschaticus, Neocaridina davidi, Cheraz quadricarinatus, Stenopus hispidus, Panulirus ornatus, Birgus latro, Scylla olivacea, or Litopenaeus vannamei. In some aspects, the aspartate 1-decarboxylase is from Egretta garzetta, Latimeria chalumnae, Coturnix japonica, Serinus canaria, Xenopus tropicalis, Nipponia nippon, Xenopus tropicalis, Daphnia magna, Phasianus colchicus, or Xenopus laevis. In some aspects, the aspartate 1-decarboxylase is an aspartate 1-decarboxylase in Table 1.

TABLE 1

| SEQ | | | | |
|---|---|---|---|---|
| ID # | Enzyme | Organism | Class | Reference ID |
| 1 | Acidic amino | Folsomia | Entognatha | A0A226ENS1 |
| | acid | candida | (Collembola) | |
| | decarboxylase | | | |
| | like-1 | | | |
| 2 | Acidic amino | Orchesella | Entognatha | A0A1D2NK65 |
| | acid | cincta | (Collembola) | |
| | decarboxylase | | | |
| | like-1 | | | |
| 3 | Hypothetical | Paralithodes | Malacostraca | DN150817_c7_g2_i3 |
| | | camtschaticus | (Crustacea) | |
| 4 | Hypothetical | Neocaridina | Malacostraca | DN596834_c0_g2_i5 |
| | | davidi | (Crustacea) | |
| 5 | Hypothetical | Cherax | Malacostraca | DN442407_c1_g1_i13 |
| | | quadricarinatus | (Crustacea) | |
| 6 | Hypothetical | Stenopus | Malacostraca | DN1193166_c6_g1_i8 |
| | | hispidus | (Crustacea) | |
| 7 | Hypothetical | Panulirus | Malacostraca | DN431573_c6_g3_i3 |
| | | ornatus | (Crustacea) | |
| 8 | Hypothetical | Birgus | Malacostraca | DN87528_c2_g1_i2 |
| | | latro | (Crustacea) | |
| 9 | Hypothetical | Scylla | Malacostraca | JAI59620.1 |
| | | olivacea | (Crustacea) | |
| 10 | Hypothetical | Litopenaeus | Malacostraca | IOCAS.LVAN21064 |
| | | vannamei | (Crustacea) | |
| 11 | Glutamate | Egretta | Aves | XP_009633445.1 |
| | decarboxylase- | garzetta | | |
| | like 1 | | | |
| 12 | Acidic amino | Latimeria | Actinistia | XP_005993764.1 |
| | acid | chalumnae | | |
| | decarboxylase | | | |
| | GADL1 | | | |
| | isoform X1 | | | |
| 13 | Acidic amino | Coturnix | Aves | XP_015709492.1 |
| | acid | japonica | | |
| | decarboxylase | | | |
| | GADL1 | | | |
| 14 | Acidic amino | Serinus | Aves | XP_030087612.1 |
| | acid | canaria | | |
| | decarboxylase | | | |
| | GADL1 | | | |
| 15 | Acidic amino | Xenopus | Amphibia | NP_001039075.1 |
| | acid | tropicalis | | |
| | decarboxylase | | | |
| | GADL1 | | | |
| 16 | Glutamate | Nipponia | Aves | XP_009464699.1 |
| | decarboxylase- | nippon | | |
| | like protein 1 | | | |
| 17 | Acidic amino | Xenopus | Amphibia | Q28D99 |
| | acid | tropicalis | | |
| | decarboxylase | | | |
| | GADL1 | | | |
| | (Fragment) | | | |
| 18 | cysteine | Daphnia | Branchiopoda | XP_032794576.1 |
| | sulfinic acid | magna | (Crustacea) | |
| | decarboxylase- | | | |
| | like isoform | | | |
| | X2 | | | |
| 19 | Acidic amino | Phasianus | Aves | XP_031446118.1 |
| | acid | colchicus | | |
| | decarboxylase | | | |
| | GADL1 | | | |
| | isoform X3 | | | |
| 20 | Acidic amino | Xenopus | Amphibia | XP_018122961.1 |
| | acid | laevis | | |
| | decarboxylase | | | |
| | GADL1 | | | |

In some aspects, the aspartate 1-decarboxylase comprises a glutamine at a residue corresponding to position 333 of the amino acid sequence of SEQ ID NO: 1, and a partial amino acid sequence having at least 75% sequence identity to amino acids 338-473 of SEQ ID NO: 1, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 338-473 of SEQ ID NO: 1. In some aspects, the aspartate 1-decarboxylase comprises a glutamine at a residue corresponding to position 378 of the amino acid sequence of SEQ ID NO: 2, and a partial amino acid sequence having at least 75% sequence identity to amino acids 383-519 of SEQ ID NO: 2, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 383-519 of SEQ ID NO: 2. In some aspects, the aspartate 1-decarboxylase comprises a glutamine at a residue corresponding to position 340 of the amino acid sequence of SEQ ID NO: 3, and a partial amino acid sequence having at least 75% sequence identity to amino acids 345-483 of SEQ ID NO: 3, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 345-483 of SEQ ID NO: 3. In some aspects, the aspartate 1-decarboxylase comprises a glutamine at a residue corresponding to position 320 of the amino acid sequence of SEQ ID NO: 4, and a partial amino acid sequence having at least 75% sequence identity to amino acids 325-457 of SEQ ID NO: 4, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 325-457 of SEQ ID NO: 4. In some aspects, the aspartate 1-decarboxylase comprises a glutamine at a residue corresponding to position 353 of the amino acid sequence of SEQ ID NO: 5, and a partial amino acid sequence having at least 75% sequence identity to amino acids 358-490 of SEQ ID NO: 5, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 358-490 of SEQ ID NO: 5. In some aspects, the aspartate 1-decarboxylase comprises a glutamine at a residue corresponding to position 320 of the amino acid sequence of SEQ ID NO: 6, and a partial amino acid sequence having at least 75% sequence identity to amino acids 325-457 of SEQ ID NO: 6, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 325-457 of SEQ ID NO: 6. In some aspects, the aspartate 1-decarboxylase comprises a glutamine at a residue corresponding to position 335 of the amino acid sequence of SEQ ID NO: 7, and a partial amino acid sequence having at least 75% sequence identity to amino acids 340-472 of SEQ ID NO: 7, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 340-472 of SEQ ID NO: 7. In some aspects, the aspartate 1-decarboxylase comprises a glutamine at a residue corresponding to position 312 of the amino acid sequence of SEQ ID NO: 8, and a partial amino acid sequence having at least 75% sequence identity to amino acids 317-453 of SEQ ID NO: 8, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 317-453 of SEQ ID NO: 8. In some aspects, the aspartate 1-decarboxylase comprises a glutamine at a residue corresponding to position 310 of the amino acid sequence of SEQ ID NO: 9, and a partial amino acid sequence having at least 75% sequence identity to amino acids 315-459 of SEQ ID NO: 9, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 315-459 of SEQ ID NO: 9. In some aspects, the aspartate 1-decarboxylase comprises a glutamine at a residue corresponding to position 380 of the amino acid sequence of SEQ ID NO: 10, and a partial amino acid sequence having at least 75% sequence identity to amino acids 385-505 of SEQ ID NO: 10, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 385-505 of SEQ ID NO: 10.

In some aspects, the aspartate 1-decarboxylase comprises an isoleucine at a residue corresponding to position 320 of the amino acid sequence of SEQ ID NO: 11, and a partial amino acid sequence having at least 75% sequence identity to amino acids 325-458 of SEQ ID NO: 11, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 325-458 of SEQ ID NO: 11. In some aspects, the aspartate 1-decarboxylase comprises an isoleucine at a residue corresponding to position 337 of the amino acid sequence of SEQ ID NO: 12, and a partial amino acid sequence having at least 75% sequence identity to amino acids 342-475 of SEQ ID NO: 12, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 342-475 of SEQ ID NO: 12. In some aspects, the aspartate 1-decarboxylase comprises an isoleucine at a residue corresponding to position 329 of the amino acid sequence of SEQ ID NO: 13, and a partial amino acid sequence having at least 75% sequence identity to amino acids 334-467 of SEQ ID NO: 13, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 334-467 of SEQ ID NO: 13. In some aspects, the aspartate 1-decarboxylase comprises an isoleucine at a residue corresponding to position 328 of the amino acid sequence of SEQ ID NO: 14, and a partial amino acid sequence having at least 75% sequence identity to amino acids 333-466 of SEQ ID NO: 14, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 333-466 of SEQ ID NO: 14. In some aspects, the aspartate 1-decarboxylase comprises an isoleucine at a residue corresponding to position 318 of the amino acid sequence of SEQ ID NO: 15, and a partial amino acid sequence having at least 75% sequence identity to amino acids 322-455 of SEQ ID NO: 15, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 322-455 of SEQ ID NO: 15. In some aspects, the aspartate 1-decarboxylase comprises an isoleucine at a residue corresponding to position 319 of the amino acid sequence of SEQ ID NO: 16, and a partial amino acid sequence having at least 75% sequence identity to amino acids 323-457 of SEQ ID NO: 16, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 323-457 of SEQ ID NO: 16. In some aspects, the aspartate 1-decarboxylase comprises an isoleucine at a residue corresponding to position 329 of the amino acid sequence of SEQ ID NO: 17, and a partial amino acid sequence having at least 75% sequence identity to amino acids 334-467 of SEQ ID NO: 17, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 334-467 of SEQ ID NO: 17. In some aspects, the aspartate 1-decarboxylase comprises an isoleucine at a residue corresponding to position 329 of the amino acid sequence of SEQ ID NO: 18, and a partial amino acid sequence having at least 75% sequence identity to amino acids 334-467 of SEQ ID NO: 18, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 334-467 of SEQ ID NO: 18. In some aspects, the aspartate 1-decarboxylase comprises an isoleucine at a residue corresponding to position 329 of the amino acid sequence of SEQ ID NO: 19, and a partial amino acid sequence having at least 75% sequence identity to amino acids 334-467 of SEQ ID NO: 19, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 334-467 of SEQ ID NO: 19. In some aspects, the aspartate 1-decarboxylase comprises an isoleucine at a residue corresponding to position 317 of the amino acid sequence of SEQ ID NO: 20, and a partial amino acid sequence having at least 75% sequence identity to amino acids 322-455 of SEQ ID NO: 20, such as at least 80% sequence identity, at least 85% sequence identity, at least 90% sequence identity, at least 95% sequence identity, at least 96% sequence identity, at least 97% sequence identity, at least 98% sequence identity, or at least 99% sequence identity to amino acids 322-455 of SEQ ID NO: 20.

In some aspects, the aspartate 1-decarboxylase has at least 70% sequence identity to the amino acid sequence of SEQ ID NO: 1, 2, 3, 4, 5, 6, 7, 8, 9, 10, 11, 12, 13, 14, 15, 16, 17, 18, 19, or 20, such as at least 80% sequence identity, at least 90% sequence identity, at least 95% sequence identity, at least 96% sequence identity, at least 97% sequence identity, at least 98% sequence identity, or at least 99% sequence identity. In some aspects, the aspartate 1-decarboxylase has 100% sequence identity to the amino acid sequence of SEQ ID NO: 1, 2, 3, 4, 5, 6, 7, 8, 9, 10, 11, 12, 13, 14, 15, 16, 17, 18, 19, or 20. In some aspects, the aspartate 1-decarboxylase comprises or consists of the amino acid sequence of SEQ ID NO: 1, 2, 3, 4, 5, 6, 7, 8, 9, 10, 11, 12, 13, 14, 15, 16, 17, 18, 19, or 20.

In some aspects, β-alanine can be converted to malonate semialdehyde by one or more polypeptide that catalyze the production of malonate semialdehyde from β-alanine, e.g., by deamination of β-alanine by a β-alanine aminotransferase or a β-alanine pyruvate aminotransferase. In some aspects, the recombinant microorganism comprises one or more 3-alanine aminotransferases including, but not limited to, enzymes with as EC number 2.6.1.-, EC number 2.6.1.18, or EC number 2.6.1.19. In some aspects, the β-alanine pyruvate aminotransferase (baat) is from Bacillus cereus. In some aspects, the β-alanine transaminase (pyd4) is from Lachancea kluyveri.

Production of 3-Hydroxypropionic Acid and Related Products from Malonate Semialdehyde

In some aspects, malonate semialdehyde can be converted to 3HP by a 3-hydroxypropionic acid dehydrogenase. In some aspects, the recombinant microorganism comprises one or more 3-hydroxypropionic acid dehydrogenases including, but not limited to, enzymes with EC number 1.1.1.-, EC number 1.1.1.298, and/or EC number 1.1.1.59. In some aspects, the 3-hydroxypropionic acid dehydrogenase (ydfg) is from Escherichia coli. In some aspects, the 3-hydroxypropionic acid dehydrogenase (mcr-1) is from Chloroflexus aurantiacus. In some aspects, the 3-hydroxypropionic acid dehydrogenase (Ydfl) is from Saccharomyces cerevisiae. In some aspects, the 3-hydroxypropionic acid dehydrogenase (Hpdl) is from Candida albicans.

In some aspects, 3HP can be converted to 3-HP-CoA by a 3-hydroxypropionyl-CoA synthetase and/or a 3-hydroxypropionyl-CoA transferase. In some aspects, the recombinant microorganism comprises one or more 3-hydroxypropionyl-CoA synthetases and/or 3-hydroxypropionyl-CoA transferases including, but not limited to, enzymes with EC number 2.8.3.1, EC number 6.2.1.17, and/or EC number 6.2.1.36. In some aspects, the 3-hydroxypropionyl-CoA transferase (pct) is from Cupriavidus necator, Clostridium propionicum, or Megasphaera elsdenii. In some aspects, the 3-hydroxypropionyl-CoA synthase (Msed 1456) is from Metallosphaera sedula. In some aspects, the 3-hydroxypropionyl-CoA synthase (Stk 07830) is from Sulfolobus tokodaii.

In some aspects, 3-HP-CoA can be converted to acrylyl-CoA by a 3-hydroxypropionyl-CoA dehydratase and/or an enoyl-CoA hydratase. In some aspects, the recombinant microorganism comprises one or more 3-hydroxypropionyl-CoA dehydratases and/or enoyl-CoA hydratases including, but not limited to, enzymes with EC number 4.2.1.116, EC number 4.2.1.55, EC number 4.2.1.150, and/or EC number 4.2.1.17. In some aspects, the 3-hydroxypropionyl-CoA dehydratase (hpcd) is from Metallosphaera sedula, Bacillus sp., or Sporanaerobacter acetigenes. In some aspects, the 3-hydroxypropionyl-CoA dehydratase is from Ruegeria pomeroyi. In some aspects, the 3-hydroxypropionyl-CoA dehydratase (St1516) is from Sulfolobus tokodaii. In some aspects, the 3-hydroxypropionyl-CoA dehydratase (Nmar_1308) is from Nitrosopumilus maritimus. In some aspects, the 3-hydroxypropionyl-CoA dehydratase (Hpcd) is from Chloroflexus aurantiacus. In some aspects, the 3-hydroxypropionyl-CoA dehydratase (Crt) is from Clostridium acetobutylicum or Clostridium pasteuranum. In some aspects, the 3-hydroxypropionyl-CoA dehydratase is from Clostridium pasteuranum. In some aspects, the 3-hydroxypropionyl-CoA dehydratase (Mels_1449) is from Megasphaera elsdenii. In some aspects, the 3-hydroxypropionyl-CoA dehydratase (Aflv_0566) is from Anoxybacillus flavithermus.

In some aspects, acrylyl-CoA can be converted to acrylic acid and/or acrylate by an acyl-CoA hydrolase and/or a thioesterase. In some aspects, the recombinant microorganism comprises one or more acyl-CoA hydrolases and/or thioesterases including, but not limited to, enzymes with EC number 3.2.1.-.

In some aspects, acrylyl-CoA can be converted to propionyl-CoA by an acrylyl-CoA reductase. In some aspects, the recombinant microorganism comprises one or more acrylyl-CoA reductases including, but not limited to, enzymes with EC number 1.3.1.84 and/or EC number 1.3.1.95. In some aspects, the acrylyl-CoA reductase (acuI) is from Ruegeria pomeroyi, Escherichia coli, or Rhodobacter sphaeroides. In some aspects, the acrylyl-CoA reductase (pcdh) is from Clostridium propionicum. In some aspects, the acrylyl-CoA reductase (acuI) is from Alcaligenes faecalis. In some aspects, the acrylyl-CoA reductase (Acr) is from Sulfolobus tokodaii. In some aspects, the acrylyl-CoA reductase (acuI) is from Escherichia coli. In some aspects, the acrylyl-CoA reductase (Acr) is from Metallosphaera sedula. In some aspects, the acrylyl-CoA reductase (Nmar_1565) is from Nitrosopumilus maritimus.

In some aspects, propionyl-CoA can be converted to propionic acid by a propionate CoA transferase. In some aspects, the recombinant microorganism comprises one or more propionate CoA transferases including, but not limited to, enzymes with EC number 2.8.3.1.

In some aspects, propionyl-CoA can be converted to propionic acid by the sequential action of a phosphotransacetylase and an acetate kinase. In some aspects, the recombinant microorganism comprises one or more phosphotransacetylases including, but not limited to, enzymes with EC number 2.3.1.-. In some aspects, the recombinant microorganism comprises one or more acetate kinases including, but not limited to, enzymes with EC number 2.7.2.1.

In some aspects, propionyl-CoA can be converted to 1-propanol by a bifunctional alcohol/aldehyde dehydrogenase, an aldehyde dehydrogenase, an alcohol dehydrogenase, or a combination thereof. In some aspects, the recombinant microorganism comprises one or more bifunctional alcohol/aldehyde dehydrogenases including, but not limited to, enzymes with EC number 1.2.1.10 and/or EC number 1.1.1.1. In some aspects, the alcohol/aldehyde dehydrogenase (adhe) is from Clostridium acetobutylicum. In some aspects, the alcohol/aldehyde dehydrogenase (adhe) is from Clostridium beijerinckii. In some aspects, the alcohol/aldehyde dehydrogenase (adhe) is from Clostridium typhimurium. In some aspects, the alcohol/aldehyde dehydrogenase (adhe) is from Clostridium arbusti. In some aspects, the alcohol/aldehyde dehydrogenase (adhE) is from Escherichia coli. In some aspects, the alcohol/aldehyde dehydrogenase (adhP) is from Escherichia coli. In some aspects, the alcohol/aldehyde dehydrogenase (bdhB) is from Clostridium acetobutylicum. In some aspects, the alcohol/aldehyde dehydrogenase (Adh2) is from Saccharomyces cerevisiae. In some aspects, the alcohol/aldehyde dehydrogenase (adhE) is from Clostridium roseum. In some aspects, the alcohol/aldehyde dehydrogenase (adhA) is from Thermoanaerobacterium saccharolyticum. In some aspects, the alcohol/aldehyde dehydrogenase (Ald6) is from Saccharomyces cerevisiae. In some aspects, the alcohol/aldehyde dehydrogenase (Aldh3AI) is from Homo sapiens. In some aspects, the recombinant microorganism comprises one or more aldehyde dehydrogenases including, but not limited to, enzymes with EC number 1.2.1.10. In some aspects, the aldehyde dehydrogenase (acetylating) (mhpf) is from Escherichia coli. In some aspects, the aldehyde dehydrogenase (acetylating) (Pdup) is from Escherichia coli. In some aspects, the aldehyde dehydrogenase (acetylating) (pdup) is from Salmonella enterica. In some aspects, the aldehyde dehydrogenase (acetylating) (aldH) is from Escherichia coli. In some aspects, the aldehyde dehydrogenase (acetylating) (ald) is from Escherichia coli. In some aspects, the recombinant microorganism comprises one or more alcohol dehydrogenases including, but not limited to, enzymes with EC number 1.1.1.1 and/or EC number 1.1.1.2. In some aspects, the alcohol dehydrogenase (alrA) is from Acinetobacter sp. In some aspects, the alcohol dehydrogenase (bdhl) is from Clostridium acetobutylicum. In some aspects, the alcohol dehydrogenase (bdhIH) is from Clostridium acetobutylicum. In some aspects, the alcohol dehydrogenase (adhA) is from Clostridium glutamicum. In some aspects, the alcohol dehydrogenase (yqhD) is from Escherichia coli. In some aspects, the alcohol dehydrogenase (adhP) is from Escherichia coli. In some aspects, the alcohol dehydrogenase (PduQ) is from Propionibacterium freudenreichii. In some aspects, the alcohol dehydrogenase (ADH1) is from Saccharomyces cerevisiae. In some aspects, the alcohol dehydrogenase (ADH2) is from Saccharomyces cerevisiae. In some aspects, the alcohol dehydrogenase (ADH4) is from Saccharomyces cerevisiae. In some aspects, the alcohol dehydrogenase (ADH6) is from Saccharomyces cerevisiae. In some aspects, the alcohol dehydrogenase (PduQ) is from Salmonella enterica. In some aspects, the alcohol dehydrogenase (Adh) is from Sulfolobus tokodaii. In some aspects, the recombinant microorganism comprises a combination of an aldehyde dehydrogenase and an alcohol dehydrogenase. In some aspects, the aldehyde dehydrogenase (acetylating) (PduP) is from Salmonella enterica and the alcohol dehydrogenase (ADH1) is from Saccharomyces cerevisiae.

In some aspects, 1-propanol can be converted to propylene by an alcohol dehydratase. In some aspects, the recombinant microorganism comprises one or more alcohol dehydratases including, but not limited to, enzymes with EC number 4.2.1.127.

In some aspects, the recombinant microorganism comprises at least one nucleic acid molecule encoding one or more polypeptides that catalyze the production from 3-HP of a product selected from 1-propanol, propionic acid, acrylic acid, butanone, 2-butanol, methyl propionate, succinic acid, 1,4-butanediol, propylene, or a combination thereof.

In some aspects, the disclosure provides a recombinant microorganism capable of producing 1-propanol comprising: (a) at least one nucleic acid molecule encoding one or more polypeptides that catalyze the production of 3-HP-CoA from 3-HP; (b) at least one nucleic acid molecule encoding one or more polypeptides that catalyze the production of acrylyl-CoA from 3-HP-CoA; (c) at least one nucleic acid molecule encoding one or more polypeptides that catalyze the production of propionyl-CoA from acrylyl-CoA; (d) at least one nucleic acid molecule encoding one or more polypeptides that catalyze the production of propionaldehyde from propionyl-CoA; and (e) at least one nucleic acid molecule encoding one or more polypeptides that catalyze the production of 1-propanol from propionaldehyde.

In some aspects, the recombinant microorganism comprises at least one nucleic acid molecule encoding: (a) a 3-hydroxypropionyl-CoA synthetase and/or a 3-hydroxypropionyl-CoA transferase, preferably wherein the 3-hydroxypropionyl-CoA synthetase and/or 3-hydroxypropionyl-CoA transferase is classified as EC number 2.8.3.1, EC number 6.2.1.17, and/or EC number 6.2.1.36; (b) a 3-hydroxypropionyl-CoA dehydratase and/or an enoyl-CoA hydratase, preferably wherein the 3-hydroxypropionyl-CoA dehydratase and/or enoyl-CoA hydratase is classified as EC number 4.2.1.116, EC number 4.2.1.55, EC number 4.2.1.150, and/or EC number 4.2.1.17; (c), an acrylyl-CoA reductase, preferably wherein the acrylyl-CoA reductase is classified as EC number 1.3.1.84 and/or EC number 1.3.1.95; and/or (d) a bifunctional alcohol/aldehyde dehydrogenase, preferably wherein the bifunctional alcohol/aldehyde dehydrogenase is classified as EC number 1.2.1.10 and/or EC number 1.1.1.1; an aldehyde dehydrogenase, preferably wherein the aldehyde dehydrogenase is classified as EC number 1.2.1.10; and/or an alcohol dehydrogenase, preferably wherein the alcohol dehydrogenase is classified as EC number 1.1.1.1 and/or EC number 1.1.1.2.

Production of Acetyl-CoA and Related Products from Malonate Semialdehyde

In some aspects, MSA can be converted to acetyl-CoA by a malonate semialdehyde dehydrogenase (acetylating). In some aspects, the recombinant microorganism comprises one or more malonate semialdehyde dehydrogenases (acetylating) including, but not limited to, enzymes with EC number 1.2.1.18. In some aspects, the malonate semialdehyde dehydrogenase (bauC) is from Pseudomonas aeruginosa. In some aspects, the malonate semialdehyde dehydrogenase (Ald6) is from Candida albicans. In some aspects, the malonate semialdehyde dehydrogenase (iolA) is from Lysteria monocytogenes. In some aspects, the malonate semialdehyde dehydrogenase (dddC) is from Halomonas sp. HTNKL. In some aspects, the malonate semialdehyde dehydrogenase is from Bacillus subtillis or Arabidopsis thaliana.

In some aspects, acetyl-CoA can be converted to acetone by the sequential action of a thiolase, a CoA transferase, and a decarboxylase. In some aspects, the recombinant microorganism comprises one or more thiolases including, but not limited to, enzymes with EC number 2.3.1.9. In some aspects, the recombinant microorganism comprises one or more CoA transferases including, but not limited to, enzymes with EC number 2.8.3.8. In some aspects, the recombinant microorganism comprises one or more decarboxylases including, but not limited to, enzymes with EC number 4.1.1.4.

In some aspects, acetone can be converted to 2-propanol by an isopropanol dehydrogenase. In some aspects, the isopropanol dehydrogenase is NAD-dependent. In some aspects, the isopropanol dehydrogenase is NADP-dependent. In some aspects, the recombinant microorganism comprises one or more isopropanol dehydrogenases including, but not limited to, enzymes with EC number 1.1.1.80. In some aspects, the recombinant microorganism comprises one or more isopropanol dehydrogenases from Candida albicans, Candida parapsilosis, Devosia riboflavina, Lactobacillus brevis and/or Clostridium beijerinckii.

In some aspects, acetyl-CoA and propionyl-CoA can be converted to methyl ethyl ketone (butanone) by the sequential actions of a beta-ketothiolase, a CoA transferase and/or a CoA hydrolase, and a decarboxylase. In some aspects, the recombinant microorganism comprises one or more beta-ketothiolases including, but not limited to, enzymes with EC number 2.3.1.16. In some aspects, the recombinant microorganism comprises one or more CoA transferases and/or CoA hydrolases including, but not limited to, enzymes with EC number 2.8.3.8. In some aspects, the recombinant microorganism comprises one or more decarboxylases including, but not limited to, enzymes with EC number 4.1.1.4. In some aspects, the enzymes used to convert propionyl-CoA and acetyl-CoA to methyl ethyl ketone are (i) a 0-ketothiolase (BktB) from Cupriavidus necator and/or a β-ketothiolase (phaA) from Acinetobacter sp., (ii) a CoA transferase (atoAD) from Escherichia coli and/or a CoA transferase (ctfAB) from Clostridium acetobutylicum, and (iii) an acetate decarboxylase (adc) from Clostridium acetobutylicum or Pseudomonas putida. Advantageously, in some aspects, the enzymes convert propionyl-CoA and acetyl-CoA into methyl ethyl ketone without formation of significant levels of undesired by-products such as acetone, thereby avoiding undesirable decreases in yield.

In some aspects, methyl ethyl ketone (MEK) can be converted into 2-butanol by an alcohol dehydrogenase (e.g., a 2-butanol dehydrogenase) or a MEK reductase. In some aspects, the alcohol dehydrogenase is NAD-dependent. In some aspects, the alcohol dehydrogenase is NADP-dependent. In some aspects, the recombinant microorganism comprises one or more alcohol dehydrogenases including, but not limited to, enzymes with EC number 1.1.1.1, EC number 1.1.1.2, EC number 1.1.1.80, or EC number 1.1.1.-. In some aspects, NAD-dependent enzymes are known as EC number 1.1.1.1. In some aspects, NADP-dependent enzymes are known as EC number 1.1.1.2. In some aspects, the 2-butanol dehydrogenase (sadh) is from Rhodococcus ruber. In some aspects, the 2-butanol dehydrogenase (adhA) is from Pyrococcus furious. In some aspects, the 2-butanol dehydrogenase (adh) is from Clostridium beijerinckii. In some aspects, the 2-butanol dehydrogenase (adh) is from Thermoanaerobacter brockii. In some aspects, the 2-butanol dehydrogenase (yqhD) is from Escherichia coli. In some aspects, the 2-butanol dehydrogenase (chnA) is from Acinetobacter sp.

In some aspects, methyl ethyl ketone can be converted to methyl propionate (and ethyl acetate) by enzymes and/or homologues that have Baeyer-Villiger monooxygenase activity. In some aspects, the recombinant microorganism comprises one or more Baeyer-Villiger monooxygenases including, but not limited to, enzymes with EC number 1.14.13.-. In an embodiment, the Baeyer-Villiger monooxygenase is from Acinetobacter calcoaceticus, Rhodococcus jostii, and/or Xanthobacter flavus.

In some aspects, 2-propanol can be converted to propylene by an alcohol dehydratase. In some aspects, the recombinant microorganism comprises one or more alcohol dehydratases including, but not limited to, enzymes with EC number 4.2.1.127.

In some aspects, the recombinant microorganism comprises at least one nucleic acid molecule encoding one or more polypeptides that catalyze the production from acetyl-CoA of a product selected from ketones, such as acetone and methyl ethyl ketone; alcohols, such as 2-propanol, 1-butanol, 2-butanol, 1,3-propanediol, isoamyl alcohol, 1,3-butanediol, 1,4-butanediol, 2,3-butanediol, and isoprenol; organic acids, such as acetic acid, butyric acid, lactic acid, adipic acid, glutamic acid, itaconic acid, caproic acid, citric acid, methacrylic acid and succinic acid; esters, such as ethyl acetate and isopropyl acetate; alkenes, such as propylene, butadiene and isoprene; amino acids, such as leucine, isoleucine, glutamine and glycine; or a combination thereof.

### EXAMPLES

### Example 1: Amino Acid Sequence Homology of SEQ ID NOs: 1-20 Compared to Aspartate 1-Decarboxylase from Aedes aegypti

Novel aspartate decarboxylase enzyme candidates were prospected and identified based on literature analysis and protein sequence homology inference considering the defined criteria of being PLP-dependent enzymes that preferentially had the glutamine or isoleucine amino acids at corresponding position 377 of the amino acid sequence from Aedes aegypti.

These novel aspartate decarboxylase enzyme candidates and homologs were identified by using techniques and methods available as known from those skilled in the Art, including the Basic Local Alignment Search Tool (BLAST) and the OrthoDB catalog. BLAST enables the identification of sequences and regions of similarity between biological sequences comparing for example nucleotide or protein sequences against databases and calculating statistical significances, while OrthoDB search similarities inside catalogs of orthologous protein-coding genes across vertebrates, arthropods, fungi, plants and bacteria. Queries used were protein sequences from Aedes aegypti and Tribolium castaneum that are available on UniProt and Gene database from NCBI with IDs Q171S0 and LOC5569335 for Aedes aegypti and IDs A7U8C7 and LOC100124592 from Tribolium castaneum, respectively.

In addition, these novel aspartate decarboxylase enzyme candidates were identified by exploring homologues inside the Arthropod phylum, as candidates from Arachnida, Crustacean and Myriapoda based on the screening of the Crustacean Annotated Transcriptome (CAT) from the Hong Kong University (http://cat.sls.cuhk.edu.hk/CRF/search), the Litopenaeus vannamei mRNA library (http://www.shrimpbase.net/vannamei.html) and the NCBI Transcriptome Shotgun Assembly (TSA). Search outcomes were manually analyzed and filtered according to previous determined criteria.

The Basic Local Alignment Search Tool (BLAST) enables the identification of regions of similarity between biological sequences by comparing them and calculating statistical significances. Sequence homology is represented by the ratio of identical amino acid residues between sequences over the total number of residues. The total and partial sequence homology percentage of SEQ ID NOs: 1-20 compared to aspartate 1-decarboxylase from Aedes aegypti, as calculated using BlastP default parameters is shown in Table 2. Partial sequence homology refers to the sequence homology of the conserved region of insect amino decarboxylases, corresponding to positions 382 to 516 of aspartate 1-decarboxylase from Aedes aegypti Q171).

TABLE 2

| | | Total sequence | Partial sequence |
|---|---|---|---|
| | | homology (%) | homology (%) |
| SEQ | | against the ADC | against the ADC |
| ID | Organism of origin/ | from Aedes | from Aedes |
| NO | source | aegypti | aegypti |
| 1 | Folsomia candida | 55 | 57 |
| 2 | Orchesella cincta | 54 | 57 |
| 3 | Paralithodes camtschaticus | 54 | 61 |
| 4 | Neocaridina davidi | 55 | 64 |
| 5 | Cheraz quadricarinatus | 57 | 61 |
| 6 | Stenopus hispidus | 56 | 64 |
| 7 | Panulirus ornatus | 58 | 64 |
| 8 | Birgus latro | 56 | 61 |
| 9 | Scylla olivacea | 53 | 55 |
| 10 | Litopenaeus vannamei | 53 | 54 |
| 11 | Egretta garzetta | 53 | 54 |
| 12 | Latimeria chalumnae | 53 | 54 |
| 13 | Coturnix japonica | 53 | 55 |
| 14 | Serinus canaria | 53 | 54 |
| 15 | Xenopus tropicalis | 55 | 54 |
| 16 | Nipponia nippon | 53 | 53 |
| 17 | Xenopus tropicalis | 55 | 54 |
| 18 | Daphnia magna | 53 | 54 |
| 19 | Phasianus colchicus | 53 | 54 |
| 20 | Xenopus laevis | 54 | 54 |

### Example 2: Aspartate 1-Decarboxylase Activity of Enzymes of SEQ ID NOs 1, 3, 4, 5, 6, 7, 8, 9, 10 and 18

Aspartate 1-decarboxylase activity of enzymes of SEQ ID NOs 1, 3, 4, 5, 6, 7, 8, 9, 10 and 18 was assessed by a phenotype-based screen of growth complementation. The nucleotide sequences corresponding to SEQ ID NOs 1, 3, 4, 5, 6, 7, 8, 9, 10 and 18 were codon-optimized according to Saccharomyces cerevisiae codon bias and cloned in a replicative plasmid for expression in yeast under the control of the weak promoter pRPLA1. A weak promoter was selected to facilitate detection of any growth improvement.

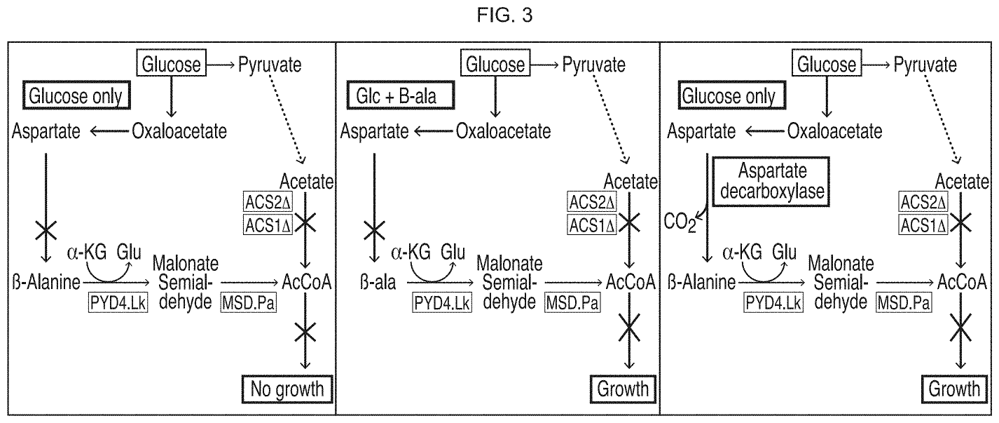

The growth complementation assay was based on the use of reporter strain YA5371-1A, having genes for the synthesis of acetyl-CoA knocked-out and expressing the β-alanine aminotransferase PYD4 from Lachancea kluyveri. This reporter strain is unable to grow in the presence of glucose as a sole carbon source because it is unable to synthesize acetyl-CoA. Expression of an active aspartate decarboxylase restores cell growth with glucose as the sole carbon source, as shown in FIG. 3.

The YA5371-1A reporter strain was independently transformed with each of the plasmids listed in Table 3 containing a different aspartate decarboxylase, with a plasmid expressing ACS2 as a positive control, and with empty plasmid as a negative control. ADC from Tribolium castaneum served as an additional positive control from the Class Insecta. The growth of the transformants was assayed on SY-U+β-alanine as a positive control and on YPDA as selective medium.

TABLE 3

| | | Common | | | |
|---|---|---|---|---|---|
| SEQ ID NO | Organism | name | Gene | Plasmid | Description |
| (̅A̅7̅U̅8̅C̅7̅)̅ | Tribolium | — | PAND.Tca | pAD4814 | pRX316-pRPLA1- |
| | castaneum | | | | PAND.Tca-tRPL15A |
| 1 | Folsomia | Springtail | PAND.Fc | pAD4873 | pRX316-pRPLA1- |
| | candida | | | | PAND.Fc-tRPL15A |
| 5 | Cherax | Redclaw | PAND.Cq | pAD4874 | pRX316-pRPLA1- |
| | quadricarinatus | crayfish | | | PAND.Cq-tRPL15A |
| 7 | Panulirus | Spiny | PAND.Po | pAD4875 | pRX316-pRPLA1- |
| | ornatus | lobster | | | PAND.Po-tRPL15A |
| 6 | Stenopus | Coral | PAND.Shi | pAD4876 | pRX316-pRPLA1- |
| | hispidus | shrimp | | | PAND.Sh-tRPL15A |
| 10 | Litopenaeus | Whiteleg | PAND.Lv | pAD4877 | pRX316-pRPLA1- |
| | vannamei | shrimp | | | PAND.Lv-tRPL15A |
| 18 | Daphnia | water flea | PAND-0.Dma | pAD5078 | pRX316-pRPLA1- |
| | magna | | | | PAND-0.Dma- |
| | | | | | tRPL15A |
| 3 | Paralithodes | Red King | PAND.Pca | pAD5079 | pRX316-pRPLA1- |
| | camtschaticus | crab | | | PAND.Pca-tRPL15A |
| 4 | Neocaridina | Cherry | PAND.Nd | pAD5080 | pRX316-pRPLA1- |
| | davidi | shrimp | | | PAND.Nd-tRPL15A |
| 8 | Birgus | Coconut | PAND-0.Bla | pAD5081 | pRX316-pRPLA1- |
| | latro | crab | | | PAND-0.Bla- |
| | | | | | tRPL15A |
| 9 | Scylla | orange | PAND.So | pAD5082 | pRX316-pRPLA1- |
| | olivacea | mud crab | | | PAND.So-tRPL15A |

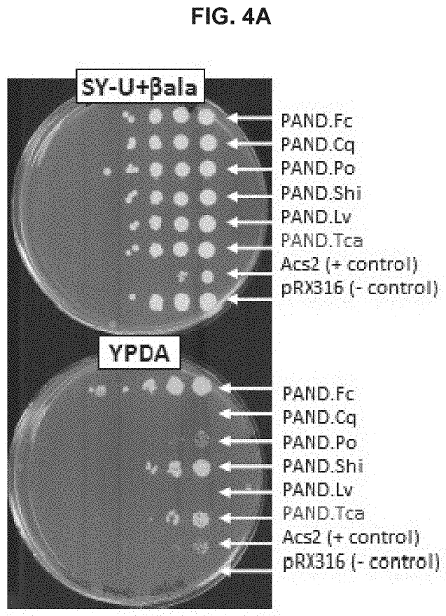

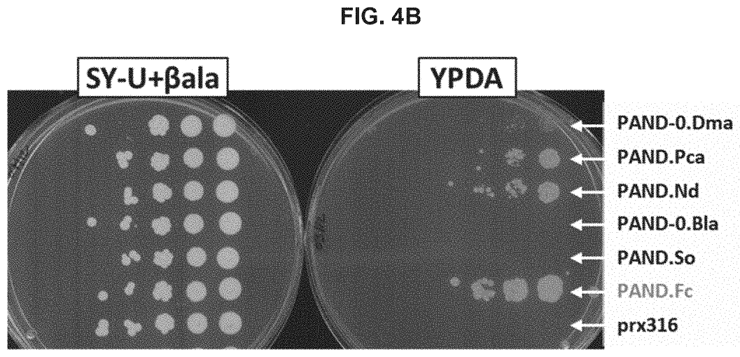

The results are provided in FIG. 4A and FIG. 4B and show that the highest level of complementation was observed with PAND.Fc, followed by PAND.Shi, PAND.Pca, PAND.Nd and then PAND.Po. PAND.Po complemented less than PAND.Tca. Under the tested assay conditions, growth was not observed for PAND.Cq, PAND.Lv, PAND-OBla and PAND.So.

### Example 3: Activity of Recombinant Yeast Strains Expressing Aspartate 1-Decarboxylases

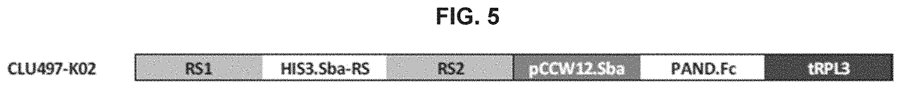

Codon-optimized nucleotide sequences corresponding to SEQ ID NOs 1, 3, 4, 5, 6, 7 and 18 were integrated into the yeast genome under the control of a strong promoter. The wild-type strain CC788-2B was transformed by the CLU497 clusters that allow the expression of the four different genes under the control of the strong promoter CCW 12, as shown in FIG. 5. As the clusters are integrated into the same genome locus, the resulting strains only differ by the expressed PAND enzyme. Obtained strains are listed in Table 4.

TABLE 4

| Strain | MAT | Genotype | Phenotype |
|---|---|---|---|
| YA5631- | MAT-a | can1-100, his3, jlp1::[HIS3.Sba-RS-PAND.Tca], leu2, | CanR, LEU-, |
| 3/4 | | trp1, ura3 | TRP-, URA- |
| YA5630- | MAT-a | can1-100, his3, jlp1::[HIS3.Sba-RS-PAND.Fc], leu2, | CanR, LEU-, |
| 1/3 | | trp1, ura3 | TRP-, URA- |
| YA5628- | MAT-a | can1-100, his3, jlp1::[HIS3.Sba-RS-PAND.Cq], leu2, | CanR, LEU-, |
| 4/5 | | trp1, ura3 | TRP-, URA- |
| YA5629- | MAT-a | can1-100, his3, jlp1::[HIS3.Sba-RS-PAND.Po], leu2, | CanR, LEU-, |
| 1/2 | | trp1, ura3 | TRP-, URA- |
| YA5632- | MAT-a | can1-100, his3, jlp1::[HIS3.Sba-RS-PAND.Shi], leu2, | CanR, LEU-, |
| 1/2 | | trp1, ura3 | TRP-, URA- |
| YA5786- | MAT-a | can1-100, his3, jlp1::[HIS3. Sba-RS-PAND-0.Dma], | CanR, LEU-, |
| 1/2 | | leu2, trp1, ura3 | TRP-, URA- |
| YA5787- | MAT-a | can1-100, his3, jlp1::[HIS3.Sba-RS-PAND.Pea], leu2, | CanR, LEU-, |
| 1/2 | | trp1, ura3 | TRP-, URA- |
| YA5788- | MAT-a | can1-100, his3, jlp1::[HIS3.Sba-RS-PAND.Nd], leu2, | CanR, LEU-, |
| 1/2 | | trp1, ura3 | TRP-, URA- |

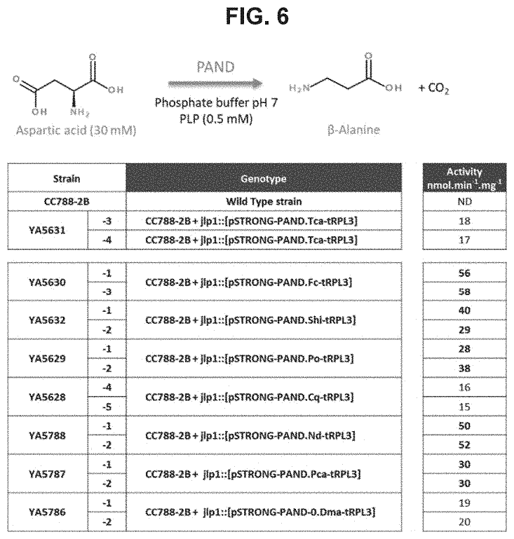

After expression, aspartate decarboxylase activity was assessed by HPLC measuring the formation of β-alanine after derivatization with an ACQ-tag. The results are provided in FIG. 6 and show that PAND.Fc, PAND.Nd, PAND.Pca and PAND.Shi were the most active aspartate decarboxylases with improved activity compared to ADC from Tribolium castaneum (PAND.Tca) from the Class Insecta. PAND.Po also demonstrated enhanced aspartate decarboxylase activity compared to PAND.Tca, while PAND.Cq and PAND-O.Dma demonstrated less or equal aspartate decarboxylase activity than PAND.Tca.

### Example 4: Production of 3-Hydroxypropionic Acid by a Recombinant Yeast Strain Expressing Aspartate 1-Decarboxylase

A recombinant yeast strain was genetically modified to produce 3-hydroxypropionic acid from glucose as a carbon source. As shown in Table 5, recombinant yeast strains had 3-hydroxypropionic acid pathway producing genes integrated into the genome, including the aspartate aminotransferase AAT from Saccharomyces cerevisiae, the β-alanine aminotransferase PYD4 from Lanchacea kluyveri, and the 3-hydroxypropionic acid dehydrogenase HPD1 from Candida albicans. The recombinant yeast strain also expressed one of the aspartate 1-decarboxylase enzymes listed in the Table 1 as described herein.

Production of 3-hydroxypropionic acid by the recombinant yeast strain was assayed after 48 hours of growth in 25 mL of rich medium (YPA) containing 8% of glucose in Erlenmeyer flasks plugged with a silicone cap with 2 pipette tips of 1 mL with filter. Stirring was maintained at 180 rpm on a 50 mm shaking diameter incubator. The 3-hydroxypropionic acid produced from glucose was measured by LC/MS-MS analysis and the results are shown in Table 5.

TABLE 5

| | | | | | | | PAND |
|---|---|---|---|---|---|---|---|
| | | OD600 | Glucose | Glycerol | EtOH | 3-HP | (nmol/min · |
| Strain | Genotype | nm | (g/L) | (g/L) | (g/L) | (g/L) | mg) |
| YA5932- | jlp1::[LEU2.Sba-RS- | 76 | ND | 2 | 28 | ND | ND |
| 2A | PYD4.Lk-PYK1], | | | | | | |
| | pyk1::[HIS3.Sba-RS- | | | | | | |
| | PEPCK-1.Ec-AAT2- | | | | | | |
| | PEPCK-1.Ec] | | | | | | |
| YA5932- | jlp1::[LEU2.Sba-RS- | 62 | 1 | 1 | 31 | 1.3 | 200 |
| 3B | PYD4.Lk-PYK1], | | | | | | |
| YA5932- | pyk1::[HIS3.Sba-RS- | 72 | 1 | 1 | 31 | 1.0 | 215 |
| 5C | PEPCK-1.Ec-AAT2- | | | | | | |
| YA5932- | PEPCK-1.Ec], | 48 | ND | 1 | 32 | 1.3 | 150 |
| 7D | ura3::[PAND.Fc-HPD1- | | | | | | |
| | 2.Cal-URA3]x5 | | | | | | |

The recombinant yeast strain expressing one of the aspartate 1-decarboxylases of the invention along with the other required 3-hydroxypropionic producing pathway genes was capable of producing 3-hydroxypropionic acid from glucose in a g/L range. In contrast, no 3-hydroxypropionic acid was produced in the absence of the aspartate 1-decarboxylase.

### Example 5: Production of 1-Propanol, Acetone, and/or 2-Propanol by a Recombinant Yeast Strain Expressing Aspartate 1-Decarboxylase

A recombinant yeast strain overexpresses at least one enzyme selected from an aspartate aminotransferase, a β-alanine aminotransferase, a 3-hydroxypropionic acid dehydrogenase, 3-hydroxypropionyl-CoA synthetase, 3-hydroxypropionyl-CoA transferase, a 3-hydroxypropionyl-CoA dehydratase, an enoyl-CoA hydratase, an acrylyl-CoA reductase, an aldehyde dehydrogenase, an alcohol dehydrogenase, a malonate semialdehyde dehydrogenase, a thiolase, a CoA transferase, an acetoacetate decarboxylase, and/or an isopropanol dehydrogenase, wherein the aspartate aminotransferase is AAT from Saccharomyces cerevisiae, the β-alanine aminotransferase is PYD4 from Lanchacea kluyveri, the 3-hydroxypropionic acid dehydrogenase is YdfG from Escherichia coli, YMR226C (YDF1) from Saccharomyces cerevisiae, or HPD1 from Candida albicans, the 3-hydroxypropionyl-CoA synthetase is a propionate CoA transferase PCT from Clostridium propionicum, the enoyl-CoA hydratase is HPCD from Ruegeria pomeroyi, the acrylyl-CoA reductase is ACR from Ruegeria pomeroyi, the propionaldehyde dehydrogenase is PDUP from Salmonella enterica, the alcohol dehydrogenase is ADH1 from Saccharomyces cerevisiae, the malonate semialdehyde dehydrogenase is MSD from Candida albicans or Pseudomonas aeruginosa, the thiolase is ERG10 from Saccharomyces cerevisiae, the CoA transferase is ATOA/ATOD from Escherichia coli, the acetoacetate decarboxylase is ADC from Paenibacillus polymyxa, and the isopropanol dehydrogenase is IPDH from Candida albicans or Clostridium beijerinckii. The recombinant yeast strain also expresses an aspartate 1-decarboxylase as described herein.

Production of 1-propanol, acetone, and/or 2-propanol by the recombinant yeast strain is assayed after 48 hours of growth in 25 mL of rich medium (YPA) containing 8% of glucose in Erlenmeyer flasks plugged with a silicone cap with 2 pipette tips of 1 mL with filter. Stirring is maintained at 180 rpm on a 50 mm shaking diameter incubator. The 1-propanol, acetone, and/or 2-propanol was measured by GC/MS-MS headspace analysis

The recombinant yeast strain expressing an aspartate 1-decarboxylase of the invention along with the 1-propanol, acetone, and/or 2-propanol pathway genes produces more 1-propanol, acetone, and/or 2-propanol in g/L compared to a recombinant yeast strain expressing the ADC from Tribolium castaneum along with the 1-propanol, acetone, and/or 2-propanol pathway genes.

Sequences
>tr|A0A226ENS1|A0A226ENS1_FOLCA Acidic amino
acid decarboxylase GADL1
OS = Folsomia candida OX = 158441
GN = Fcan01_06240 PE = 3 SV = 1
SEQ ID NO: 1
MGDAKVSENGTEKVSESKVLTENPKYYKTYPEREVHEKFFRDVFEIILKD
ALFEGIQRDQPVVRFEQPHDLWKILNLKLGRDPAHNHDVLLDLVKDVIKY
SVKTGHPYFINQLYSGIDPYGVAAEWVASALNGSVYTYEVAPVFTLMELE
VFERMRNIIGFPKNQGDGLFCPGGSLANGYAISVARYKKRPEIKELGLSG
VKPMIMFVSEDAHYSFKKLASFQGIGLKNVVGVKVDSRGKMNVTELDKEI
QSSIDKGFDPFLVSATAGTTVLGAFDPIDEIAEVCKKYGLWLHVDGAWGG
GALMSDTYRDLMKGIEKADSVTWNPHKLLCAPQQCSTFLLKDGTIATDAH
ATRATYLFQQDKFYEAQFDTGDKHIQCGRRADVLKFWLMWKAKGKDGFEA
HIDHIFGLAKFCVEELRRRGPSFKLLLEDPECTNVVFWYIPPSLQDMNQS
SPEFWDRIHKIAPKIKERMMRQGTMMCTYQPLRQYQNFFRVVIQSSEVNE
KDVTYFLDEIEKCGKDL
>tr|A0A1D2NK65|A0A1D2NK65_ORCCI Cysteine
sulfinic acid decarboxylase OS = Orchesella
cincta OX = 48709 GN = Ocin01_00983 PE = 4
SV = 1
SEQ ID NO: 2
MKHQWFKVSSYSWSNTQTKRVCQAATPNPNYKLIGIMGSNKSESGEELQD
NPLYYKTFPSKDLHEKFFHDVFDLILKEAMFEGVQRDKLVVRFKLPENLE
KILHLKLGKKPVKSQEDLLDLLKEVMKYSVKTGHPYFINQLFSGLDPYGI
CAEWVASALNSSVYTYEVAPVFTLMELEVFEKMRDMVGFPRGQGDGLFCP
GGSMANGYAISVARYRKRPEVKERGLCGMKEMVIFVSEDCHYSFKKLASF
QGHYYCYYLILCFTLPPKGLGMKNVVGIKVDHKGKMIIEDLVDKIEISIE
KGQDPFMVAATAGTTVLGAFDPIEEVAKVCQKYELYLHVDAAWGGGALMS
PKYRHLLKGIEKADSVTWNPHKLLCAPQQCSTFLIKDSDVCTQTHATKAT
YLFQQDKFYDAAYYDTGDKHVQCGRRADVLKFWFMWKAKGSEGFAAHIDH
IFGVAEYCVQELRSRSPAFQLLLEEPECTNITFWYIPPSLRDMNNKSDEF
WEKLHKVAPKIKEAMMKRGSMMITYQPLRNYQNFFRLVIQSSEVTKEDIK
YFLDEIENCGKDIEVKMQDILLLVKYPYGKRTCAFLHTLYFNIFLPMLIF
FINCTPIPDDISNQNTTSTDNISTAASASSAGHPSSANQTVFVGDICGQN
MFWDPTLNTCKPKPNAGKSGVLDATNNNASNTNATIAKPGKKKKPFHSEE
EVTDPPHHHETETNKPHVNETKQIRVDTNSDEEDVIPVKTDPHCGLGYEW
SADDMMCAKDEQ
>TRINITY_DN150817_c7_g2_i3, Paralithodes
camtschaticus (Red king crab)
SEQ ID NO: 3
LPATLQGLPKRSVAQLHNERMADQTNDKVWKSLECSWESGPDKNHHLHFL
QSLLNMLLDQAVFKSTDRNSKLVEWMSPEQLADQVDLQLGEKGITQGQLM
TIVEDVVKYSVKTGHPYFVNQLFSSLDVYGLVGQWVTDALNPSVYTYEVA
PVFTLMEIQVLEAMGKYVGYDEQDGLFSPGGSISNMYGMLLARHHAFPNA
KKTGMSQLGRLVVFTSQDAHYSLAKASVTLGLGSDNLVLVDVDQRGRMDV
DHLKKCIRKTREEGATPIMVCATAGTTVIGAYDPINSIADVCEHEKIWLH
VDAAWGGGALVSRTYRHKLTGVHRSDSLTWNPHKLLASPQQCSVFLTRHP
GLLKQCNSASAAYLFQKDKFYDTGYDTGDKHLQCGRRADVMKFWCMWKAK
GSEGLERHIDHIFAMSELFTDKIRNREGFKLLLEPECTNVCFHYLPPSLR
CSSGGGGGSGGSNCDRLHVVAPKVKERMVKGGRMMVTYQPLRSHPNFFRL
VLQNSQVGPQDIDYFINTIEELAADL
>TRINITY_DN596834_c0_g2_i5, Neocaridina
davidi (Cherry shrimp)
SEQ ID NO: 4
MNNEEASSNSLGRLCPWESGPDQSLHPHFLSESLQLLIDHAVFGGTDRST
KVVEWVEPDDLKKRLNLTLEDKGVTQAELLRCLKDVVKYSVKTGHPYFIN
QLFSSLDVYGLVGQWVTDALNPSVYTYEVAPVFTLMEIEVMNMMASFVGY
KEHDGLFSPGGSMSNMYGMLLARYYRFPQVKKRGISGIGRLVAFTSVDAH
YSSAKAAMTMGIGADNLVLVNVDDEGRMDPEHLKQCIAKARQEGGIPFVV
VATAGTTVLGAYDPINSIADVCQAEGLWFHCDAAWGGGALMSNKHKGKLS
GIHRADSITWNPHKMLAAPQQCSLFLTKHIGLLKECNSASAAYLFQKDKF
YDTSYDTGDKHLQCGRRADVLKFWTMWKAKGTSGLERHIDHLFEMSDLFT
ETIRKREGFRLVIEPQCTNVCFWYEPPSLRSKRSNPQYNTLLNAVAPRIK
ERMVKTGTMMITYQPLRGNPNFFRLVLQNSKVNADDIRYFADQIEELGKD
L
>TRINITY_DN442407_c1_g1_i13, Cheraz
quadricarinatus (Redclaw crayfish)
SEQ ID NO: 5
SRALTGQSSCGRRGRMNNREDTSNGGKSEKNDASGGKYEDNNLGKKCGWE
SGPEAAHHAHFLRAVLDLLVEKAVFQGTDRSSKVVEWVEPSELQQRLQLD
LGDDGVTQGDLLRHLDQVVRYSVKTGHPYFINQLFSSLDVYGLVGQWVTD
ALNPSVYTYEVSPVFTLMEIKVLSAMADLVGYKEHDGLFCPGGSISNMYG
MLLARYHHCPDVKKRGLHGLGHQLVVLTSADSHYSLLKSAMTLGLGADNL
LPVDVDEAGRMDVGHLRQRIKSAREEGAVPFMVCATAGTTVLGAYDPLDA
VAEVCGDESLWLHVDAAWGGGALLSSRHKHKLLGIHRADSVTWNPHKLLA
SPQQCSVFLTSHPGLLDACNSASANYLFQKDKFYDTSYDVGDKHLQCGRR
VDVLKFWTMWKAKGTTGLEKHVERVFEMSQLFADKIRFREGFKLLLEPEC
TNVCFWYEPPSLRTKRAHPEYNKLLNAVAPKIKERMVKTGSMMITYQPLR
GQPNFFRLVLQNSQVNEHDINYFIAQIEHLGRDL
>TRINITY_DN1193166_c6_g1_i8, Stenopus
hispidus (Coral shrimp)
SEQ ID NO: 6
MGEESNSKNGLGQACSWESGPDPIRHSHFLKEVLQLLVDHAVFQATDRSN
KVVEWVEPNELKKRMNLQLEEEGVTQGVLLGCLKDVVKYSVKTGHPYFIN
QLFSSLDVYGLVGQWVTDALNPSVYTYEVAPVFTLMEIEVLSAMASYIGY
TQHDGLFSPGGSMSNMYGMLLARHHRFPEVKKHGIGGLGRLVAFTSIDAH
YSLKKAAVTLGLGSDSLVLINVDDAGRMDVGHLKESIRRVRQEGAIPFMV
CATTGTTVLGAYDPISAIADVCEAEGLWLHADAAWGGGALISKKYKYKLH
GIHRADSVTWNPHKMLASPQQCSVFLTRHVGLLNSCNSASAAYLFQKDKF
YDTSYDTGDKHLQCGRRADVLKFWTMWKAKGTKGLEQHVDRLFEMSIAFA
DIIRAREGFSLVSEPECTNVCFWYEPPSLRAKPAHPEYKTLLNAVAPCIK
ERMVKSGTMMVTYQPLREHPNFFRLVLQNSQVNHEDIKYFVDQIDRLGRD
L
>TRINITY_DN431573_c6_83_i3, Panulirus
ornatus (Spiny lobster)
SEQ ID NO: 7
MEKQEENGLGVAYAPGNGPDESENGVWGACSWQSGPDHAKHEHFLKAVLH
LLVEEAVFRATDRHSKVVEWMEPDQLRKRLDLTLREEGVTQDDLLKQLKD
VVKYSVKTGHPYFINQLFSSLDVYGLVGQWITDALNPSVYTYEVAPVFTL
MEFQVLATMADHVGYKEHDGLFSPGGSMSNLYGMLLARYSHFPDVKKRGT
NGLGRLVVFTSVDAHYSLAKSAMTLGLGSDNLILINVNHDGRMDVNHLKE
SISKAREEGATPFMVCATAGTTVLGAYDPVDAVADVCQDEDLWLHVDAAW
GGGALLSPRYKHKLKGIHRADSVTWNPHKLLAAPQQCSVFLTRHVGLLKE
CNSASAAYLFQKDKFYDTSYDTGDKHLQCGRRADVLKFWTMWKAKGTKGL
EKHIERLFEMSEAFVKQIRDREGFVLLMEPQCTNVCFWYEPPSLRGQRCH
PNYHTRLHAVAPLIKERMVKTGSMMVTYQPLREKPNFFRLVLQNSQVNHD
DIHYFVRQIEALGSDL
>TRINITY_DN87528_c2_g1_i2, Birgus
latro (Coconut crab)
SEQ ID NO: 8
MDNNALCTWRSGPVREMHFEFLKSIFEMLVEEAVFNGTARTSKVVEWQDP
EELKAKIDFGVREAGMSHSALMALMRNVVKYSVKTGHPYFINQLFSSLDV
YGLVGQWVTDALNPSVYTYEVAPVFTLMEIEVVAAMGKYVGYRKQDGLFS
PGGSISNMYGMILARYNAFPQSKKTGISQLGRLVVFTSVDAHYSLAKSAV
VLGLGSDNLVLVDVDESGKMDINHLEKCIKEVKQEGGRPIMVCATAGTTV
LGAYDPINPIADICERERIWLHVDAAWGGGALVSSIHRHKLDGIHRADSV
TWNPHKLLASPQQCSIFLTRHTGVLKECNAASAAYLFQKDKFYDTAYDTG
DKHLQCGRRADVMKFWTMWKAKGSVGLEKHINHIFATSKQFADKIKNRAG
FHLILEPECTNVCFLYHPPSLRSGGSCGRSDGGSWERLHKIAPKLKERMV
KEGRMMLTYQPLRHYPNFFRLVLQNSQVVEEDVDYFIRTIEELAAD
>JAI59620.1 hypothetical protein
PF00282.15, Scylla olivacea
SEQ ID NO: 9
MEEKCGWETGPNRVQHETFLKAVLDMLLEKAVFDGTNRKNKLVEWQEPEE
LKQKMSLAVREEGMTHGELFALMQQVVKYSVKTGHPYFINQLYSGLDVYG
LVGQWVTDALNPSVYTYEVAPVFTLMEIEVLSAMASLVGFEQHDGLFSPG
GSISNMYGMLLARYRTFPEIKSKGCSELGRLVALTSIDAHYSLKKAAMTL
GLGSDNLVLVNTDAVGRMDVNHLKHCIEEEKKKKSTIIMVCATAGTTVLG
AYDPVAAIADVCEKEGIWLHVDAAWGGGALISPALRHKIRGIHRADSVTW
NPHKLLVAPQQCSVFLTRHPGLLKACNSASAAYLFQKDKFYNTSYDTGDQ
HLQCGRRADVLKFWAMWKAKGTSGLAQHMERVFSLAEEFAGMVSRRGRGW
RLLQIPECTNVCFWYLPSALHDVPAAVLQGTLTTPADHQHFKRVSAVAPR
LKERMVREGRMMITYQPLRGRPNFFRLVLQSSQVTSRDLEYFINTIEELA
QNSQE
>IOCAS.LVAN21064, Litopenaeus vannamei
SEQ ID NO: 10
MQLHFLSTPRKKAPLPLPAAASCVHGAAVSRRGAAVCSLLRREWKASSSG
AVVETCPSCGSVCAAAAAMSSQDENGLGAPCDWRSGPDIRHHSVFLKETL
QLLVENAVFQATDRNNKVVEWVEPEDLKKQLDLRLGDEGITHATLLRYLR
GVIRYSVKTGHPYFINQLFSSLDVYGLVGQWVTDALNPSVYTYEVAPVFT
IMENEVLANMASIVGYSQHDGLFAPGGSMANMYGMLLARHRRFPEVKRSG
VGGLGRLVAFTSVDAHYSNTKSAMTLGLGSDNLVLVNVDEEGRMDVDHLK
ECIARTKQDGAIPFLVTATAGRHNRPRAYDPLDAIADGQRRDVATPTPGE
DPSVPQAKATNIHRSDSVTWNPHKLLAAPQQCSVFLTRHLGLLTQCNSAS
APYLFQKDKFYDTKYDVGDKHLQCGRRADGTKGLERHIDHLFEMTKFFTD
TIRDREGFRLVLEPQCTNVCFWYERLAAGKRAHEQYPQLLNSVAPRIKER
MVKTGTMMITYQPLHSRPNFFRLVLQNSQVNAEDMKYFANQFEVLGRDL
>XP_009633445.1 PREDICTED: glutamate
decarboxylase-like protein 1
[Egretta garzetta]
SEQ ID NO: ll
MLQKKKNAVLVDGVILNGPIMDSKAGEKFVEEACKIIMEEVIQKADDVTG
KVCEWRAPETLKQILDLEMRDTGESHQKLLQLCRDVIQYSVKTSHPRFFN
QLYAGIDYYSLVARFITEALNPSVYTYEVSPVFLLVEEAVIKKMIEFIGW
EEGDGIFNPGGSVSNMYAMNLARYKFCPEIKEKGLSGLPRLVLFTSEECH
YSMKKAASFLGIGTENVYFIKTDERGKMIPEELEKQVQRARKEGSAPFLV
CATAGTTVLGAFDPLDKIADICEKHGLWLHVDASWGGSALISRKHRRLLH
GIHRADSVAWNPHKMLLAGIQCCALLVKDNSGLLKKCYSAKASYLFQQDK
FYDVSYDTGDKSIQCSRRPDAFKFWLMWKALGTTGLEERVNRALALARYL
VEEIKKREGFQLLLEPEYANVCFWYIPPSLRKMEDGPEFWQKLHQVAPII
KERMMKKGSMMLGYQPHQGKVNFFRQVVISPQVSREDMDFLLDEIELLAK
DL
>XP_005993764.1 PREDICTED: acidic amino
acid decarboxylase GADL1 isoform X1
[Latimeria chalumnae]
SEQ ID NO: 12
MMSTIICNGAQTVAQNKEQLEKKNAILVDGVILNGPIIDAKAGQQFIQEA
FPIIMEEAIRKGTDVNEKVCEWQPPAQLKKILDLELRDVGENHQRLLQRC
QDVIRYSVKTSHPRFYNQLYAGMDPYSLVARFVTEAVNPSVYTYEVSPVF
VLMEEAVLKKMIEHVGWKEGDGIFSPGGSVSNMYAVNVARYKFCPDIKEK
GLSGMPRLVMFTSEECHYSVKKAAAFLGIGTQNVYVVKADDRGKMIPEEL
EKQIEQAKKEGALPFLVSATAGTTVLGAFDPLDKVAGICERHGLWFHVDA
AWGGSALMSRKHRHILQGIHRADSVAWNPHKMLMAGIQCCAFLVKGNTGL
LKECHSACASYLFQQDKFYDVEYDIGDKSIQCSRRADAFKFWLMWKAIGT
RGLEERVNRAFALARYLADEIKKREEFRLILEPEYASICFWYIPPSLRNM
EEGPEFWQKLNKVAPIVKERMMKKGSMMVGYQPHRGKVNFFRQIIISPQV
SREDLDFLLNEIDNLGKDL
>XP_015709492.1 acidic amino acid
decarboxylase GADL1 [Coturnix japonica]
SEQ ID NO: 13
MEANTCKQDVLQKKKNAILVDGVILNGPITDSKAGEKFVEEACKIIMEEI
IQKADDVTEKVCEWRAPETLKKILDLEMRDTGESHQKLLQLCQDVIQYSV
KTNHPRFFNQLYAGIDYYSLVARFITEALNPSVYTYEVSPVFLLVEEAVI
KKMIEFIGWEDGDGIFNPGGSVSNMYAMNLARYKFCPEIKEKGLSGLPRL
VLFASEECHYSMKKAASFLGIGTENVYFVKTDERGKMIPEELEKQVQRAR
KEGSAPFLVCATAGTTVLGAFDPLDKIADICEKHDLWLHVDASWGGSALI
SRKHRKLLHGIQRADSVAWNPHKMLLAGIQCCALLVKDNSGLLKKCYSAK
AAYLFQQDKFYDVSYDTGDKSIQCSRRPDAFKFWLMWKALGTTGLEERVN
RALALARYLVEEIKKREGFQLLLEPEYANVCFWYVPPSLRKMEDGPEFWQ
KLHQVAPVVKERMMKKGSMMLGYQPNQGKVNFFRQVVISPQVSREDMDFL
LDEIELLAKDL
>XP_030087612.1 acidic amino acid
decarboxylase GADL1 [Serinus canaria]
SEQ ID NO: 14
MEVGVCKQEMLQKKNAVLVDGVILNGPIMDSKAGEKFVEEACKIIMEEVV
QKADDVTEKVCEWQAPEKLKQILDLEMRDTGECHQKILQLCRDVIKYSVK
TNHPRFFNQLYAGIDYYSLVARFITEALNPSVYTYEVSPVFLLVEEAVIK
KMIEFIGWEEGDGIFNPGGSISNMYAMNLARYKFCPEIKEKGLSSLPRLV
LFASEECHYSMKKAASFLGIGTENVYFIKTDERGKMIPEELEKQVQRARK
EGSAPFLVCATAGTTVLGAFDPLDKIADICEKHGLWLHVDASWGGSALIS
RKHRRLLHGIHRADSVAWNPHKMLLAGIQCCALLVKDNSGLLKKCYSAEA
AYLFQQDKFYDVSYDTGDKSIQCSRRPDAFKFWLMWKALGTAGLEQRVNR
ALALARYLVEEIKKREGFQLLLEPEYANVCFWYIPPSLRKMEDGPEFWQK
LHQVAPIIKERMMKKGSMMLGYQPHWGKVNFFRQVVISPQVSREDMDFLL
DEIELLAKDL
>NP_001039075.1 acidic amino acid
decarboxylase GADL1 [Xenopus tropicalis]
SEQ ID NO: 15
MKRNAVLVDGVVLNGPIIDSSSGEKFVEDVYRILMNELVYKASDINQKVC
EWQEPEQLKKLLDLNIKDNGEPHEKLLQLCKNVIKYSVKTSHPRFFNQLY
AGMDHYSLAARFITEALNPSVYTYEVSPVFILTEEAILKKMIEFLGWKEG
DGIFSPGGSVSNMYAVNLARYKYCPDIKQKGLSSAPRLVMFTSEECHYSM
KKAAAFLGIGTENVYFVKTDDRGKMIPEELENQIQRAKKEGAVPFLVSAT
SGTTVLGAFDPLDDIANICEKHKLWFHVDASWGGSALMSQKYRKRLHGIH
RADSVAWNPHKMLMAGIQCCALLVRDNSGLLKRCHSAEATYLFQQDKFYD
VQYDTGDKSIQCSRRADAFKFWMMWKALGTTGLEERINRALALTRYLASE
IKKRDGFELLWEPEYANTCFWYIPPSFRNMEKGPEYWRKFSNVAPTIKER
MMKKGSMMVGYQPHRDKVNFFRHIVISPQVSREDMDFVLDEIERLGRDL
>XP_009464699.1 PREDICTED: glutamate
decarboxylase-like protein 1 [Nipponia nippon]
SEQ ID NO: 16
MLQKKNAVLVDGVILNGPIMDSKAGEKFVEEACKIIMEEVIQKANDVTAK
VCEWQAPETLKQILDLEMRDTGESHQKLLQVCRDVIRYSVKTGHPRFFNQ
LYAGIDYYSVVARFITEALNPSVYTYEVSPVFLLVEEAVIKKMIEFIGWE
EGDGIFNPGGSVSNMYAMNLARYKFCPEIKAKGLSGLPRLVLFTSEECHY
SMKKAASFLGIGTENVYFLKTDERGKMIPEELEKQVQRARKEGSAPFLVC
ATAGTTVLGAFDPLDKIADICEKHGLWLHVDASWGGSALISRKHRKLLHG
IHRADSVAWNPHKMLLAGIQCCAFLVKDNSGLLKKCYSAKAAYLFQQDKF
YDVSYDTGDKSIQCSRRPDAFKFWLMWKALGTTGLEERVNRALALARYLV
EEIKKREGFRLLLEPEYANVCFWYIPPSLRKMEDGPEFWQKLHQVAPIIK
ERMMKKGSMMLGYQPHQGKVNFFRQVVISPQVSREDMDFLLDEIELLAKD
L
>sp|Q28D99|GADL1_XENTR Acidic amino acid
decarboxylase GADL1 (Fragment)
OS = Xenopus tropicalis OX = 8364
GN = gadl1 PE = 2 SV = 2
SEQ ID NO: 17
HDIDKNKQETRPMKRNAVLVDGVVLNGPIIDSSSGEKFVEDVYRILMNEL
VYKASDINQKVCEWQEPEQLKKLLDLNIKDNGEPHEKLLQLCKNVIKYSV
KTSHPRFFNQLYAGMDHYSLAARFITEALNPSVYTYEVSPVFILTEEAIL
KKMIEFLGWKEGDGIFSPGGSVSNMYAVNLARYKYCPDIKQKGLSSAPRL
VMFTSEECHYSMKKAAAFLGIGTENVYFVKTDDRGKMIPEELENQIQRAK
KEGAVPFLVSATSGTTVLGAFDPLDDIANICEKHKLWFHVDASWGGSALM
SQKYRKRLHGIHRADSVAWNPHKMLMAGIQCCALLVRDNSGLLKRCHSAE
ATYLFQQDKFYDVQYDTGDKSIQCSRRADAFKFWMMWKALGTTGLEERIN
RALALTRYLASEIKKRDGFELLWEPEYANTCFWYIPPSFRNMEKGPEYWR
KFSNVAPTIKERMMKKGSMMVGYQPHRDKVNFFRHIVISPQVSREDMDFV
LDEIERLGRDL
>XP_032852946.1 acidic amino acid
decarboxylase GADL1 isoform X1
[Tyto alba alba]
SEQ ID NO: 18
MEVGVCKQEMLQKKKNAVLVDGVILNGPIMDSKAGEKFVEEACKIIMEEV
IQKADDVTEKVCEWRAPETLKQILDLEMRDTGETHQKLLQLCRDVIQYSV
KTGHPRFFNQLYAGIDYYSLVARFITEALNPSVYTYEVSPVFLLVEEAVI
KKMIEFIGWEEGDGIFNPGGSVSNMYAMNLARYKFFPEIKEKGLSGLPRL
VLFTSEECHYSMKKAASFLGIGTENVYFIKTDERGKMIPEELEKQVQRAR
KEGSAPFLVCATAGTTVLGAFDPLDKIADICEKHGLWLHVDASWGGSALI
SRKHCRLLQGIHRADSVAWNPHKMLLAGIQCCALLVKDNSGLLKKCYSAK
AAYLFQQDKFYDVSYDTGDKSIQCSRRPDAFKFWLMWKALGTTGLEQRVN
RALALARYLVEEIKKREGFQLLLEPEYANVCFWYIPPSLRKMEDGPEFWQ
KLHQVAPIIKERMMKKGSMMLGYQPHQDKVNFFRQVVISPHVSREDMDFL
LDEIELLAKDL
>XP_031446118.1 acidic amino acid
decarboxylase GADL1 isoform X3
[Phasianus colchicus]
SEQ ID NO: 19
MEASTCKQDVLQRKKNAVLVDGVILNGPITDSKAGEKFVEEACKIIMEEI
IQKADDVTEKVCEWRDPETLKKILDLEMRDTGESHQKLLQLCQDVIQYSV
KTNHPRFFNQLYAGIDYYSLVARFITEALNPSVYTYEVSPVFLLVEEAVI
KKMIEFIGWEEGDGIFNPGGSVSNMYAMNLARYKFCPEIKEKGLSGLPRL
VLFTSEECHYSMKKAASFLGIGTENVYFVKTDERGKMIPEELEKQVQRAR
KEGSAPFLVCATAGTTVLGAFDPLDKIADICEKYDLWLHVDASWGGSALI
SRKHRRLLRGIQRADSVAWNPHKMLLAGIQCCALLVKDNSGLLKKCYSAK
AAYLFQQDKFYDVSYDTGDKSIQCSRRPDAFKFWLMWKALGTTGLEERVN
RALALARYLVEEIKKREGFQLLLEPEYANVCFWYIPPSLRKMEDGPEFWQ
KLHRVAPVVKERMMKKGSMMLGYQPNQGKVNFFRQVVISPQVSREDMDFL
LDEIELLAKDL
>XP_018122961.1 PREDICTED: acidic amino
acid decarboxylase GADL1 [Xenopus laevis]
SEQ ID NO: 20
MKRNAVLVDGVVLNGPIIDSSSGEKFVEDVYRILMNELVYKASDVNQKVC
EWQEPDQLKKLLDLNIKDNGEPHEKLLQLCQNVIKYSVKTSHPRFFNQLY
AGMDHYSLAARFITEALNPSVYTYEVSPVFILTEEAILMKMIEFIGWKEG
DGIFSPGGSVSNMYAVNLARYKCCPDIKQKGLSSAPRLVMFTSEECHYSM
KKAAAFLGIGTENVYFVKTDDRGKMIPEELENQIQRAKKEGAVPFLVSAT
SGTTVLGAFDPLDNIANICEKHKLWFHVDASWGGSALMSRKYRKCLHGIH
RADSVAWNPHKMLMAGIQCCALLVKDNSGLLKRCHSAEATYLFQQDKFYD
VQYDTGDKSVQCSRRADAFKFWMMWKALGTTGLEERINRALALTRYLASE
IKKRDGFELLWEPEYANTCFWYIPPSFRNMEKGPEYWKNFSKVAPTIKER
MMKKGSMMVGYQPHRDKVNFFRHIVISPQVSREDMDFVLDEIERLGRDL

1. A recombinant microorganism comprising:
(a) at least one nucleic acid molecule encoding an aspartate 1-decarboxylase that catalyzes the production of β-alanine from aspartate; and
(b) at least one nucleic acid molecule encoding one or more polypeptides that catalyze the production of malonate semialdehyde (MSA) from β-alanine;
wherein the aspartate 1-decarboxylase is of the Class Malacostraca, Entognatha, Amphibia, Aves, or Actinistia.

2. The recombinant microorganism of claim 1, wherein the aspartate 1-decarboxylase is of the Class Malacostraca or Entognatha, wherein the aspartate 1-decarboxylase comprises: (a) a glutamine at a residue corresponding to position 333 of the amino acid sequence of SEQ ID NO: 1, and a partial amino acid sequence having at least 75% sequence identity to amino acids 338-473 of SEQ ID NO: 1; (b) a glutamine at a residue corresponding to position 378 of the amino acid sequence of SEQ ID NO: 2, and a partial amino acid sequence having at least 75% sequence identity to amino acids 383-519 of SEQ ID NO: 2; (c) a glutamine at a residue corresponding to position 340 of the amino acid sequence of SEQ ID NO: 3, and a partial amino acid sequence having at least 75% sequence identity to amino acids 345-483 of SEQ ID NO: 3; (d) a glutamine at a residue corresponding to position 320 of the amino acid sequence of SEQ ID NO: 4, and a partial amino acid sequence having at least 75% sequence identity to amino acids 325-457 of SEQ ID NO: 4; (e) a glutamine at a residue corresponding to position 353 of the amino acid sequence of SEQ ID NO: 5, and a partial amino acid sequence having at least 75% sequence identity to amino acids 358-490 of SEQ ID NO: 5; (f) a glutamine at a residue corresponding to position 320 of the amino acid sequence of SEQ ID NO: 6, and a partial amino acid sequence having at least 75% sequence identity to amino acids 325-457 of SEQ ID NO: 6; (g) a glutamine at a residue corresponding to position 335 of the amino acid sequence of SEQ ID NO: 7, and a partial amino acid sequence having at least 75% sequence identity to amino acids 340-472 of SEQ ID NO: 7; (h) a glutamine at a residue corresponding to position 312 of the amino acid sequence of SEQ ID NO: 8, and a partial amino acid sequence having at least 75% sequence identity to amino acids 317-453 of SEQ ID NO: 8; (i) a glutamine at a residue corresponding to position 310 of the amino acid sequence of SEQ ID NO: 9, and a partial amino acid sequence having at least 75% sequence identity to amino acids 315-459 of SEQ ID NO: 9; or (j) a glutamine at a residue corresponding to position 380 of the amino acid sequence of SEQ ID NO: 10, and a partial amino acid sequence having at least 75% sequence identity to amino acids 385-505 of SEQ ID NO: 10.

3. The recombinant microorganism of claim 1, wherein the aspartate 1-decarboxylase is of the Class Amphibia, Aves, or Actinistia, wherein the aspartate 1-decarboxylase comprises: (a) an isoleucine at a residue corresponding to position 320 of the amino acid sequence of SEQ ID NO: 11, and a partial amino acid sequence having at least 75% sequence identity to amino acids 325-458 of SEQ ID NO: 11; (b) an isoleucine at a residue corresponding to position 337 of the amino acid sequence of SEQ ID NO: 12, and a partial amino acid sequence having at least 75% sequence identity to amino acids 342-475 of SEQ ID NO: 12; (c) an isoleucine at a residue corresponding to position 329 of the amino acid sequence of SEQ ID NO: 13, and a partial amino acid sequence having at least 75% sequence identity to amino acids 334-467 of SEQ ID NO: 13; (d) an isoleucine at a residue corresponding to position 328 of the amino acid sequence of SEQ ID NO: 14, and a partial amino acid sequence having at least 75% sequence identity to amino acids 333-466 of SEQ ID NO: 14; (e) an isoleucine at a residue corresponding to position 318 of the amino acid sequence of SEQ ID NO: 15, and a partial amino acid sequence having at least 75% sequence identity to amino acids 322-455 of SEQ ID NO: 15; (f) an isoleucine at a residue corresponding to position 319 of the amino acid sequence of SEQ ID NO: 16, and a partial amino acid sequence having at least 75% sequence identity to amino acids 323-457 of SEQ ID NO: 16; (g) an isoleucine at a residue corresponding to position 329 of the amino acid sequence of SEQ ID NO: 17, and a partial amino acid sequence having at least 75% sequence identity to amino acids 334-467 of SEQ ID NO: 17; (h) an isoleucine at a residue corresponding to position 329 of the amino acid sequence of SEQ ID NO: 18, and a partial amino acid sequence having at least 75% sequence identity to amino acids 334-467 of SEQ ID NO: 18; (i) an isoleucine at a residue corresponding to position 329 of the amino acid sequence of SEQ ID NO: 19, and a partial amino acid sequence having at least 75% sequence identity to amino acids 334-467 of SEQ ID NO: 19; or (j) an isoleucine at a residue corresponding to position 317 of the amino acid sequence of SEQ ID NO: 20, and a partial amino acid sequence having at least 75% sequence identity to amino acids 322-455 of SEQ ID NO: 20.

4. The recombinant microorganism of claim 1, wherein the aspartate 1-decarboxylase is from Folsomia candida, Orchesella cincta, Paralithodes camtschaticus, Neocaridina davidi, Cheraz quadricarinatus, Stenopus hispidus, Panulirus ornatus, Birgus latro, Scylla olivacea, or Litopenaeus vannamei.

5. The recombinant microorganism of claim 1, wherein the aspartate 1-decarboxylase is from Egretta garzetta, Latimeria chalumnae, Coturnix japonica, Serinus canaria, Xenopus tropicalis, Nipponia nippon, Xenopus tropicalis, Daphnia magna, Phasianus colchicus, or Xenopus laevis.

6. The recombinant microorganism of claim 1, wherein the polypeptide that catalyzes the production of MSA from β-alanine is a β-alanine pyruvate amino transferase and/or a β-alanine transaminase, preferably wherein the β-alanine pyruvate amino transferase and/or a β-alanine transaminase is classified as EC number 2.6.1.-, EC number 2.6.1.19, and/or EC number 2.6.1.18.

7. The recombinant microorganism of claim 1, further comprising at least one nucleic acid molecule encoding one or more polypeptides that catalyze the production of 3-hydroxypropionic acid (3-HP) from malonate semialdehyde.

8. The recombinant microorganism of claim 7, wherein the polypeptide that catalyzes the production of 3-HP from MSA is a 3-hydroxypropionic acid dehydrogenase, preferably wherein the 3-hydroxypropionic acid dehydrogenase is classified as EC number 1.1.1.-, EC number 1.1.1.298, and/or EC number 1.1.1.59.

9. The recombinant microorganism of claim 7, further comprising at least one nucleic acid molecule encoding one or more polypeptides that catalyze the production of a derivative selected from 1-propanol, propionic acid, acrylic acid, butanone, 2-butanol, methyl propionate, succinic acid, 1,4-butanediol, propylene, or a combination thereof from 3-HP.

10. The recombinant microorganism of claim 7, wherein the microorganism is capable of producing 1-propanol, further comprising:
(a) at least one nucleic acid molecule encoding one or more polypeptides that catalyze the production of 3-HP-CoA from 3-HP;
(b) at least one nucleic acid molecule encoding one or more polypeptides that catalyze the production of acrylyl-CoA from 3-HP-CoA;
(c) at least one nucleic acid molecule encoding one or more polypeptides that catalyze the production of propionyl-CoA from acrylyl-CoA;
(d) at least one nucleic acid molecule encoding one or more polypeptides that catalyze the production of propionaldehyde from propionyl-CoA; and
(e) at least one nucleic acid molecule encoding one or more polypeptides that catalyze the production of 1-propanol from propionaldehyde.

11. The recombinant microorganism of claim 10, wherein microorganism comprises at least one nucleic acid molecule encoding: (a) a 3-hydroxypropionyl-CoA synthetase and/or a 3-hydroxypropionyl-CoA transferase, preferably wherein the 3-hydroxypropionyl-CoA synthetase and/or 3-hydroxypropionyl-CoA transferase is classified as EC number 2.8.3.1, EC number 6.2.1.17, and/or EC number 6.2.1.36; (b) a 3-hydroxypropionyl-CoA dehydratase and/or an enoyl-CoA hydratase, preferably wherein the 3-hydroxypropionyl-CoA dehydratase and/or enoyl-CoA hydratase is classified as EC number 4.2.1.116, EC number 4.2.1.55, EC number 4.2.1.150, and/or EC number 4.2.1.17; (c), an acrylyl-CoA reductase, preferably wherein the acrylyl-CoA reductase is classified as EC number 1.3.1.84 and/or EC number 1.3.1.95; and/or (d) a bifunctional alcohol/aldehyde dehydrogenase, preferably wherein the bifunctional alcohol/aldehyde dehydrogenase is classified as EC number 1.2.1.10 and/or EC number 1.1.1.1; an aldehyde dehydrogenase, preferably wherein the aldehyde dehydrogenase is classified as EC number 1.2.1.10; and/or an alcohol dehydrogenase, preferably wherein the alcohol dehydrogenase is classified as EC number 1.1.1.1 and/or EC number 1.1.1.2.

12. The recombinant microorganism of claim 7, further comprising:
(a) at least one nucleic acid molecule encoding one or more polypeptides that catalyze the production of 3-HP-CoA from 3-HP; and
(b) at least one nucleic acid molecule encoding one or more polypeptides that catalyze the production of acrylyl-CoA from 3-HP-CoA.

13. The recombinant microorganism of claim 12, wherein the polypeptide that catalyzes the production of 3-HP-CoA from 3-HP is a 3-hydroxypropionyl-CoA synthetase and/or a 3-hydroxypropionyl-CoA transferase, preferably wherein the 3-hydroxypropionyl-CoA synthetase and/or 3-hydroxypropionyl-CoA transferase is classified as EC number 2.8.3.1, EC number 6.2.1.17, and/or EC number 6.2.1.36.

14. The recombinant microorganism of claim 12, wherein the polypeptide that catalyzes the production of acrylyl-CoA from 3-HP-CoA is a 3-hydroxypropionyl-CoA dehydratase and/or an enoyl-CoA hydratase, preferably wherein the 3-hydroxypropionyl-CoA dehydratase and/or enoyl-CoA hydratase is classified as EC number 4.2.1.116, EC number 4.2.1.55, EC number 4.2.1.150, and/or EC number 4.2.1.17.

15. The recombinant microorganism of claim 12, further comprising at least one nucleic acid molecule encoding one or more polypeptides that catalyze the production of acrylic acid and/or acrylate from acrylyl-CoA.

16. The recombinant microorganism of claim 15, wherein the polypeptide that catalyzes the production of acrylic acid and/or acrylate from acrylyl-CoA is an acyl-CoA hydrolase and/or a thioesterase, preferably wherein the acyl-CoA hydrolase and/or thioesterase is classified as EC number 3.2.1.-.

17. The recombinant microorganism of claim 12, further comprising at least one nucleic acid molecule encoding one or more polypeptides that catalyze the production of propionyl-CoA from acrylyl-CoA.

18. The recombinant microorganism of claim 17, wherein the polypeptide that catalyzes the production of propionyl-CoA from acrylyl-CoA is an acrylyl-CoA reductase, preferably wherein the acrylyl-CoA reductase is classified as EC number 1.3.1.84 and/or EC number 1.3.1.95.

19. The recombinant microorganism of claim 17, further comprising at least one nucleic acid molecule encoding one or more polypeptides that catalyze the production of propionic acid from propionyl-CoA.

20. The recombinant microorganism of claim 19, wherein the polypeptide that catalyzes the production of propionic acid from propionyl-CoA is a propionate CoA transferase, preferably wherein the propionate CoA transferase is classified as EC number 2.8.3.1.

21. The recombinant microorganism of claim 19, wherein the polypeptides that catalyze the production of propionic acid from propionyl-CoA are:
(a) a phosphotransacetylase, preferably wherein the phosphotransacetylase is classified as EC number 2.3.1.-.; and
(b) an acetate kinase, preferably wherein the acetate kinase is classified as EC number 2.7.2.1.

22. The recombinant microorganism of claim 17, further comprising at least one nucleic acid molecule encoding one or more polypeptides that catalyze the production of 1-propanol from propionyl-CoA.

23. The recombinant microorganism of claim 22, wherein the polypeptide that catalyzes the production of 1-propanol from propionyl-CoA is a bifunctional alcohol/aldehyde dehydrogenase, preferably wherein the bifunctional alcohol/aldehyde dehydrogenase is classified as EC number 1.2.1.10 and/or EC number 1.1.1.1; an aldehyde dehydrogenase, preferably wherein the aldehyde dehydrogenase is classified as EC number 1.2.1.10; and/or an alcohol dehydrogenase, preferably wherein the alcohol dehydrogenase is classified as EC number 1.1.1.1 and/or EC number 1.1.1.2.

24. The recombinant microorganism of claim 1, further comprising at least one nucleic acid molecule encoding one or more polypeptides that catalyze the production of acetyl-CoA from MSA.

25. The recombinant microorganism of claim 24, wherein the polypeptide that catalyzes the production of acetyl-CoA from MSA is a malonate semialdehyde dehydrogenase (acetylating), preferably wherein the malonate semialdehyde dehydrogenase (acetylating) is classified as EC number 1.2.1.18.

26. The recombinant microorganism of claim 24, further comprising at least one nucleic acid molecule encoding one or more polypeptides that catalyze the production of a derivative selected from ketones, such as acetone and methyl ethyl ketone; alcohols, such as 2-propanol, 1-butanol, 2-butanol, 1,3-propanediol, isoamyl alcohol, 1,3-butanediol, 1,4-butanediol, 2,3-butanediol, and isoprenol; organic acids, such as acetic acid, butyric acid, lactic acid, adipic acid, glutamic acid, itaconic acid, caproic acid, citric acid, methacrylic acid and succinic acid; esters, such as ethyl acetate and isopropyl acetate; alkenes, such as propylene, butadiene and isoprene; amino acids, such as leucine, isoleucine, glutamine and glycine; or a combination thereof from acetyl-CoA.

27. The recombinant microorganism of claim 24, further comprising at least one nucleic acid molecule encoding one or more polypeptides that catalyze the production of acetone from acetyl-CoA.

28. The recombinant microorganism of claim 27, wherein the polypeptides that catalyze the production of acetone from acetyl-CoA are:
(a) a thiolase, preferably wherein the thiolase is classified as EC number 2.3.1.9;
(b) a CoA transferase, preferably wherein the CoA transferase is classified as EC number 2.8.3.8; and
(c) a decarboxylase, preferably wherein the decarboxylase is classified as EC number 4.1.1.4.

29. The recombinant microorganism of claim 27, further comprising at least one nucleic acid molecule encoding one or more polypeptides that catalyze the production of 2-propanol (isopropanol) from acetone.

30. The recombinant microorganism of claim 29, wherein the polypeptide that catalyzes the production of 2-propanol from acetone is an isopropanol dehydrogenase, preferably wherein the isopropanol dehydrogenase is classified as EC number 1.1.1.80.

31. The recombinant microorganism of claim 17, further comprising at least one nucleic acid molecule encoding one or more polypeptides that catalyze the production of methyl ethyl ketone from the condensation of acetyl-CoA and propionyl-CoA.

32. The recombinant microorganism of claim 31, wherein the polypeptides that catalyze the production of methyl ethyl ketone from the condensation of acetyl-CoA and propionyl-CoA sequentially are:
(a) a beta-ketothiolase, preferably wherein the beta-ketothiolase is classified as EC number 2.3.1.16;
(b) a CoA transferase and/or a CoA hydrolase, preferably wherein the CoA transferase and/or a CoA hydrolase is classified as EC number 2.8.3.8; and
(c) a decarboxylase, preferably wherein the decarboxylase is classified as EC number 4.1.1.4.

33. The recombinant microorganism of claim 9, further comprising at least one nucleic acid molecule encoding one or more polypeptides that catalyze the production of propylene from 1-propanol and/or 2-propanol, wherein the polypeptide is an alcohol dehydratase, preferably wherein the alcohol dehydratase is classified as EC number 4.2.1.127.

34. The recombinant microorganism of claim 1, wherein the aspartate 1-decarboxylase uses pyridoxal-5′-phosphate (PLP) as a cofactor.

35. (canceled)

36. (canceled)

37. (canceled)

38. The recombinant microorganism of claim 1, wherein the aspartate 1-decarboxylase has at least 95% sequence identity to the amino acid sequence of SEQ ID NO: 1, 2, 3, 4, 5, 6, 7, 8, 9, 10, 11, 12, 13, 14, 15, 16, 17, 18, 19, or 20.

39. The recombinant microorganism of claim 1, wherein the aspartate 1-decarboxylase has 100% sequence identity to the amino acid sequence of SEQ ID NO: 1, 2, 3, 4, 5, 6, 7, 8, 9, 10, 11, 12, 13, 14, 15, 16, 17, 18, 19, or 20.

40. The recombinant microorganism of claim 1, wherein the microorganism is selected from a bacterium, a fungus, or a yeast.

41. A method of producing MSA comprising: contacting the recombinant microorganism of claim 1 with a fermentable carbon source under conditions sufficient to produce MSA.

42. The method of claim 41, wherein the recombinant microorganism further produces 3-HP, acrylic acid, propionic acid, 1-propanol, acetone, isopropanol (2-propanol), butanone, 1-butanol, 2-butanol, methyl propionate, 1,3-propanediol, isoamyl alcohol, 1,3-butanediol, 1,4-butanediol, 2,3-butanediol, lactic acid, adipic acid, glutamic acid, itaconic acid, ethyl acetate, isopropyl acetate, acetic acid, butyric acid, caproic acid, citric acid, methacrylic acid, succinic acid, propylene, butadiene, ethanol, isoprenol, leucine, isoleucine, glutamine, glycine, isoprene, or a combination thereof.


SEQUENCE LISTING (20 sequences)

SEQ ID NO: 1 - protein, 517 aa, Folsomia candida
MGDAKVSENGTEKVSESKVLTENPKYYKTYPEREVHEKFFRDVFEIILKDALFEGIQRDQPVVRFEQPHDLWKILNLKLGRDPAHNHDVLLDLVKDVIKYSVKTGHPYFINQLYSGIDPYGVAAEWVASALNGSVYTYEVAPVFTLMELEVFERMRNIIGFPKNQGDGLFCPGGSLANGYAISVARYKKRPEIKELGLSGVKPMIMFVSEDAHYSFKKLASFQGIGLKNVVGVKVDSRGKMNVTELDKEIQSSIDKGFDPFLVSATAGTTVLGAFDPIDEIAEVCKKYGLWLHVDGAWGGGALMSDTYRDLMKGIEKADSVTWNPHKLLCAPQQCSTFLLKDGTIATDAHATRATYLFQQDKFYEAQFDTGDKHIQCGRRADVLKFWLMWKAKGKDGFEAHIDHIFGLAKFCVEELRRRGPSFKLLLEDPECTNVVFWYIPPSLQDMNQSSPEFWDRIHKIAPKIKERMMRQGTMMCTYQPLRQYQNFFRVVIQSSEVNEKDVTYFLDEIEKCGKDL

SEQ ID NO: 2 - protein, 762 aa, Orchesella cincta
MKHQWFKVSSYSWSNTQTKRVCQAATPNPNYKLIGIMGSNKSESGEELQDNPLYYKTFPSKDLHEKFFHDVFDLILKEAMFEGVQRDKLVVRFKLPENLEKILHLKLGKKPVKSQEDLLDLLKEVMKYSVKTGHPYFINQLFSGLDPYGICAEWVASALNSSVYTYEVAPVFTLMELEVFEKMRDMVGFPRGQGDGLFCPGGSMANGYAISVARYRKRPEVKERGLCGMKEMVIFVSEDCHYSFKKLASFQGHYYCYYLILCFTLPPKGLGMKNVVGIKVDHKGKMIIEDLVDKIEISIEKGQDPFMVAATAGTTVLGAFDPIEEVAKVCQKYELYLHVDAAWGGGALMSPKYRHLLKGIEKADSVTWNPHKLLCAPQQCSTFLIKDSDVCTQTHATKATYLFQQDKFYDAAYYDTGDKHVQCGRRADVLKFWFMWKAKGSEGFAAHIDHIFGVAEYCVQELRSRSPAFQLLLEEPECTNITFWYIPPSLRDMNNKSDEFWEKLHKVAPKIKEAMMKRGSMMITYQPLRNYQNFFRLVIQSSEVTKEDIKYFLDEIENCGKDIEVKMQDILLLVKYPYGKRTCAFLHTLYFNIFLPMLIFFINCTPIPDDISNQNTTSTDNISTAASASSAGHPSSANQTVFVGDICGQNMFWDPTLNTCKPKPNAGKSGVLDATNNNASNTNATIAKPGKKKKPFHSEEEVTDPPHHHETETNKPHVNETKQIRVDTNSDEEDVIPVKTDPHCGLGYEWSADDMMCAKDEQ

SEQ ID NO: 3 - protein, 526 aa, Paralithodes camtschaticus
LPATLQGLPKRSVAQLHNERMADQTNDKVWKSLECSWESGPDKNHHLHFLQSLLNMLLDQAVFKSTDRNSKLVEWMSPEQLADQVDLQLGEKGITQGQLMTIVEDVVKYSVKTGHPYFVNQLFSSLDVYGLVGQWVTDALNPSVYTYEVAPVFTLMEIQVLEAMGKYVGYDEQDGLFSPGGSISNMYGMLLARHHAFPNAKKTGMSQLGRLVVFTSQDAHYSLAKASVTLGLGSDNLVLVDVDQRGRMDVDHLKKCIRKTREEGATPIMVCATAGTTVIGAYDPINSIADVCEHEKIWLHVDAAWGGGALVSRTYRHKLTGVHRSDSLTWNPHKLLASPQQCSVFLTRHPGLLKQCNSASAAYLFQKDKFYDTGYDTGDKHLQCGRRADVMKFWCMWKAKGSEGLERHIDHIFAMSELFTDKIRNREGFKLLLEPECTNVCFHYLPPSLRCSSGGGGGSGGSNCDRLHVVAPKVKERMVKGGRMMVTYQPLRSHPNFFRLVLQNSQVGPQDIDYFINTIEELAADL

SEQ ID NO: 4 - protein, 501 aa, Neocaridina davidi
MNNEEASSNSLGRLCPWESGPDQSLHPHFLSESLQLLIDHAVFGGTDRSTKVVEWVEPDDLKKRLNLTLEDKGVTQAELLRCLKDVVKYSVKTGHPYFINQLFSSLDVYGLVGQWVTDALNPSVYTYEVAPVFTLMEIEVMNMMASFVGYKEHDGLFSPGGSMSNMYGMLLARYYRFPQVKKRGISGIGRLVAFTSVDAHYSSAKAAMTMGIGADNLVLVNVDDEGRMDPEHLKQCIAKARQEGGIPFVVVATAGTTVLGAYDPINSIADVCQAEGLWFHCDAAWGGGALMSNKHKGKLSGIHRADSITWNPHKMLAAPQQCSLFLTKHIGLLKECNSASAAYLFQKDKFYDTSYDTGDKHLQCGRRADVLKFWTMWKAKGTSGLERHIDHLFEMSDLFTETIRKREGFRLVIEPQCTNVCFWYEPPSLRSKRSNPQYNTLLNAVAPRIKERMVKTGTMMITYQPLRGNPNFFRLVLQNSKVNADDIRYFADQIEELGKDL

SEQ ID NO: 5 - protein, 534 aa, Cheraz quadricarinatus
SRALTGQSSCGRRGRMNNREDTSNGGKSEKNDASGGKYEDNNLGKKCGWESGPEAAHHAHFLRAVLDLLVEKAVFQGTDRSSKVVEWVEPSELQQRLQLDLGDDGVTQGDLLRHLDQVVRYSVKTGHPYFINQLFSSLDVYGLVGQWVTDALNPSVYTYEVSPVFTLMEIKVLSAMADLVGYKEHDGLFCPGGSISNMYGMLLARYHHCPDVKKRGLHGLGHQLVVLTSADSHYSLLKSAMTLGLGADNLLPVDVDEAGRMDVGHLRQRIKSAREEGAVPFMVCATAGTTVLGAYDPLDAVAEVCGDESLWLHVDAAWGGGALLSSRHKHKLLGIHRADSVTWNPHKLLASPQQCSVFLTSHPGLLDACNSASANYLFQKDKFYDTSYDVGDKHLQCGRRVDVLKFWTMWKAKGTTGLEKHVERVFEMSQLFADKIRFREGFKLLLEPECTNVCFWYEPPSLRTKRAHPEYNKLLNAVAPKIKERMVKTGSMMITYQPLRGQPNFFRLVLQNSQVNEHDINYFIAQIEHLGRDL

SEQ ID NO: 6 - protein, 501 aa, Stenopus hispidus
MGEESNSKNGLGQACSWESGPDPIRHSHFLKEVLQLLVDHAVFQATDRSNKVVEWVEPNELKKRMNLQLEEEGVTQGVLLGCLKDVVKYSVKTGHPYFINQLFSSLDVYGLVGQWVTDALNPSVYTYEVAPVFTLMEIEVLSAMASYIGYTQHDGLFSPGGSMSNMYGMLLARHHRFPEVKKHGIGGLGRLVAFTSIDAHYSLKKAAVTLGLGSDSLVLINVDDAGRMDVGHLKESIRRVRQEGAIPFMVCATTGTTVLGAYDPISAIADVCEAEGLWLHADAAWGGGALISKKYKYKLHGIHRADSVTWNPHKMLASPQQCSVFLTRHVGLLNSCNSASAAYLFQKDKFYDTSYDTGDKHLQCGRRADVLKFWTMWKAKGTKGLEQHVDRLFEMSIAFADIIRAREGFSLVSEPECTNVCFWYEPPSLRAKPAHPEYKTLLNAVAPCIKERMVKSGTMMVTYQPLREHPNFFRLVLQNSQVNHEDIKYFVDQIDRLGRDL

SEQ ID NO: 7 - protein, 516 aa, Panulirus ornatus
MEKQEENGLGVAYAPGNGPDESENGVWGACSWQSGPDHAKHEHFLKAVLHLLVEEAVFRATDRHSKVVEWMEPDQLRKRLDLTLREEGVTQDDLLKQLKDVVKYSVKTGHPYFINQLFSSLDVYGLVGQWITDALNPSVYTYEVAPVFTLMEFQVLATMADHVGYKEHDGLFSPGGSMSNLYGMLLARYSHFPDVKKRGTNGLGRLVVFTSVDAHYSLAKSAMTLGLGSDNLILINVNHDGRMDVNHLKESISKAREEGATPFMVCATAGTTVLGAYDPVDAVADVCQDEDLWLHVDAAWGGGALLSPRYKHKLKGIHRADSVTWNPHKLLAAPQQCSVFLTRHVGLLKECNSASAAYLFQKDKFYDTSYDTGDKHLQCGRRADVLKFWTMWKAKGTKGLEKHIERLFEMSEAFVKQIRDREGFVLLMEPQCTNVCFWYEPPSLRGQRCHPNYHTRLHAVAPLIKERMVKTGSMMVTYQPLREKPNFFRLVLQNSQVNHDDIHYFVRQIEALGSDL

SEQ ID NO: 8 - protein, 496 aa, Birgus latro
MDNNALCTWRSGPVREMHFEFLKSIFEMLVEEAVFNGTARTSKVVEWQDPEELKAKIDFGVREAGMSHSALMALMRNVVKYSVKTGHPYFINQLFSSLDVYGLVGQWVTDALNPSVYTYEVAPVFTLMEIEVVAAMGKYVGYRKQDGLFSPGGSISNMYGMILARYNAFPQSKKTGISQLGRLVVFTSVDAHYSLAKSAVVLGLGSDNLVLVDVDESGKMDINHLEKCIKEVKQEGGRPIMVCATAGTTVLGAYDPINPIADICERERIWLHVDAAWGGGALVSSIHRHKLDGIHRADSVTWNPHKLLASPQQCSIFLTRHTGVLKECNAASAAYLFQKDKFYDTAYDTGDKHLQCGRRADVMKFWTMWKAKGSVGLEKHINHIFATSKQFADKIKNRAGFHLILEPECTNVCFLYHPPSLRSGGSCGRSDGGSWERLHKIAPKLKERMVKEGRMMLTYQPLRHYPNFFRLVLQNSQVVEEDVDYFIRTIEELAAD

SEQ ID NO: 9 - protein, 505 aa, Scylla olivacea
MEEKCGWETGPNRVQHETFLKAVLDMLLEKAVFDGTNRKNKLVEWQEPEELKQKMSLAVREEGMTHGELFALMQQVVKYSVKTGHPYFINQLYSGLDVYGLVGQWVTDALNPSVYTYEVAPVFTLMEIEVLSAMASLVGFEQHDGLFSPGGSISNMYGMLLARYRTFPEIKSKGCSELGRLVALTSIDAHYSLKKAAMTLGLGSDNLVLVNTDAVGRMDVNHLKHCIEEEKKKKSTIIMVCATAGTTVLGAYDPVAAIADVCEKEGIWLHVDAAWGGGALISPALRHKIRGIHRADSVTWNPHKLLVAPQQCSVFLTRHPGLLKACNSASAAYLFQKDKFYNTSYDTGDQHLQCGRRADVLKFWAMWKAKGTSGLAQHMERVFSLAEEFAGMVSRRGRGWRLLQIPECTNVCFWYLPSALHDVPAAVLQGTLTTPADHQHFKRVSAVAPRLKERMVREGRMMITYQPLRGRPNFFRLVLQSSQVTSRDLEYFINTIEELAQNSQE

SEQ ID NO: 10 - protein, 549 aa, Litopenaeus vannamei
MQLHFLSTPRKKAPLPLPAAASCVHGAAVSRRGAAVCSLLRREWKASSSGAVVETCPSCGSVCAAAAAMSSQDENGLGAPCDWRSGPDIRHHSVFLKETLQLLVENAVFQATDRNNKVVEWVEPEDLKKQLDLRLGDEGITHATLLRYLRGVIRYSVKTGHPYFINQLFSSLDVYGLVGQWVTDALNPSVYTYEVAPVFTIMENEVLANMASIVGYSQHDGLFAPGGSMANMYGMLLARHRRFPEVKRSGVGGLGRLVAFTSVDAHYSNTKSAMTLGLGSDNLVLVNVDEEGRMDVDHLKECIARTKQDGAIPFLVTATAGRHNRPRAYDPLDAIADGQRRDVATPTPGEDPSVPQAKATNIHRSDSVTWNPHKLLAAPQQCSVFLTRHLGLLTQCNSASAPYLFQKDKFYDTKYDVGDKHLQCGRRADGTKGLERHIDHLFEMTKFFTDTIRDREGFRLVLEPQCTNVCFWYERLAAGKRAHEQYPQLLNSVAPRIKERMVKTGTMMITYQPLHSRPNFFRLVLQNSQVNAEDMKYFANQFEVLGRDL

SEQ ID NO: 11 - protein, 502 aa, Egretta garzetta
MLQKKKNAVLVDGVILNGPIMDSKAGEKFVEEACKIIMEEVIQKADDVTGKVCEWRAPETLKQILDLEMRDTGESHQKLLQLCRDVIQYSVKTSHPRFFNQLYAGIDYYSLVARFITEALNPSVYTYEVSPVFLLVEEAVIKKMIEFIGWEEGDGIFNPGGSVSNMYAMNLARYKFCPEIKEKGLSGLPRLVLFTSEECHYSMKKAASFLGIGTENVYFIKTDERGKMIPEELEKQVQRARKEGSAPFLVCATAGTTVLGAFDPLDKIADICEKHGLWLHVDASWGGSALISRKHRRLLHGIHRADSVAWNPHKMLLAGIQCCALLVKDNSGLLKKCYSAKASYLFQQDKFYDVSYDTGDKSIQCSRRPDAFKFWLMWKALGTTGLEERVNRALALARYLVEEIKKREGFQLLLEPEYANVCFWYIPPSLRKMEDGPEFWQKLHQVAPIIKERMMKKGSMMLGYQPHQGKVNFFRQVVISPQVSREDMDFLLDEIELLAKDL

SEQ ID NO: 12 - protein, 519 aa, Latimeria chalumnae
MMSTIICNGAQTVAQNKEQLEKKNAILVDGVILNGPIIDAKAGQQFIQEAFPIIMEEAIRKGTDVNEKVCEWQPPAQLKKILDLELRDVGENHQRLLQRCQDVIRYSVKTSHPRFYNQLYAGMDPYSLVARFVTEAVNPSVYTYEVSPVFVLMEEAVLKKMIEHVGWKEGDGIFSPGGSVSNMYAVNVARYKFCPDIKEKGLSGMPRLVMFTSEECHYSVKKAAAFLGIGTQNVYVVKADDRGKMIPEELEKQIEQAKKEGALPFLVSATAGTTVLGAFDPLDKVAGICERHGLWFHVDAAWGGSALMSRKHRHILQGIHRADSVAWNPHKMLMAGIQCCAFLVKGNTGLLKECHSACASYLFQQDKFYDVEYDIGDKSIQCSRRADAFKFWLMWKAIGTRGLEERVNRAFALARYLADEIKKREEFRLILEPEYASICFWYIPPSLRNMEEGPEFWQKLNKVAPIVKERMMKKGSMMVGYQPHRGKVNFFRQIIISPQVSREDLDFLLNEIDNLGKDL

SEQ ID NO: 13 - protein, 511 aa, Coturnix japonica
MEANTCKQDVLQKKKNAILVDGVILNGPITDSKAGEKFVEEACKIIMEEIIQKADDVTEKVCEWRAPETLKKILDLEMRDTGESHQKLLQLCQDVIQYSVKTNHPRFFNQLYAGIDYYSLVARFITEALNPSVYTYEVSPVFLLVEEAVIKKMIEFIGWEDGDGIFNPGGSVSNMYAMNLARYKFCPEIKEKGLSGLPRLVLFASEECHYSMKKAASFLGIGTENVYFVKTDERGKMIPEELEKQVQRARKEGSAPFLVCATAGTTVLGAFDPLDKIADICEKHDLWLHVDASWGGSALISRKHRKLLHGIQRADSVAWNPHKMLLAGIQCCALLVKDNSGLLKKCYSAKAAYLFQQDKFYDVSYDTGDKSIQCSRRPDAFKFWLMWKALGTTGLEERVNRALALARYLVEEIKKREGFQLLLEPEYANVCFWYVPPSLRKMEDGPEFWQKLHQVAPVVKERMMKKGSMMLGYQPNQGKVNFFRQVVISPQVSREDMDFLLDEIELLAKDL

SEQ ID NO: 14 - protein, 510 aa, Serinus canaria
MEVGVCKQEMLQKKNAVLVDGVILNGPIMDSKAGEKFVEEACKIIMEEVVQKADDVTEKVCEWQAPEKLKQILDLEMRDTGECHQKILQLCRDVIKYSVKTNHPRFFNQLYAGIDYYSLVARFITEALNPSVYTYEVSPVFLLVEEAVIKKMIEFIGWEEGDGIFNPGGSISNMYAMNLARYKFCPEIKEKGLSSLPRLVLFASEECHYSMKKAASFLGIGTENVYFIKTDERGKMIPEELEKQVQRARKEGSAPFLVCATAGTTVLGAFDPLDKIADICEKHGLWLHVDASWGGSALISRKHRRLLHGIHRADSVAWNPHKMLLAGIQCCALLVKDNSGLLKKCYSAEAAYLFQQDKFYDVSYDTGDKSIQCSRRPDAFKFWLMWKALGTAGLEQRVNRALALARYLVEEIKKREGFQLLLEPEYANVCFWYIPPSLRKMEDGPEFWQKLHQVAPIIKERMMKKGSMMLGYQPHWGKVNFFRQVVISPQVSREDMDFLLDEIELLAKDL

SEQ ID NO: 15 - protein, 499 aa, Xenopus tropicalis
MKRNAVLVDGVVLNGPIIDSSSGEKFVEDVYRILMNELVYKASDINQKVCEWQEPEQLKKLLDLNIKDNGEPHEKLLQLCKNVIKYSVKTSHPRFFNQLYAGMDHYSLAARFITEALNPSVYTYEVSPVFILTEEAILKKMIEFLGWKEGDGIFSPGGSVSNMYAVNLARYKYCPDIKQKGLSSAPRLVMFTSEECHYSMKKAAAFLGIGTENVYFVKTDDRGKMIPEELENQIQRAKKEGAVPFLVSATSGTTVLGAFDPLDDIANICEKHKLWFHVDASWGGSALMSQKYRKRLHGIHRADSVAWNPHKMLMAGIQCCALLVRDNSGLLKRCHSAEATYLFQQDKFYDVQYDTGDKSIQCSRRADAFKFWMMWKALGTTGLEERINRALALTRYLASEIKKRDGFELLWEPEYANTCFWYIPPSFRNMEKGPEYWRKFSNVAPTIKERMMKKGSMMVGYQPHRDKVNFFRHIVISPQVSREDMDFVLDEIERLGRDL

SEQ ID NO: 16 - protein, 501 aa, Nipponia nippon
MLQKKNAVLVDGVILNGPIMDSKAGEKFVEEACKIIMEEVIQKANDVTAKVCEWQAPETLKQILDLEMRDTGESHQKLLQVCRDVIRYSVKTGHPRFFNQLYAGIDYYSVVARFITEALNPSVYTYEVSPVFLLVEEAVIKKMIEFIGWEEGDGIFNPGGSVSNMYAMNLARYKFCPEIKAKGLSGLPRLVLFTSEECHYSMKKAASFLGIGTENVYFLKTDERGKMIPEELEKQVQRARKEGSAPFLVCATAGTTVLGAFDPLDKIADICEKHGLWLHVDASWGGSALISRKHRKLLHGIHRADSVAWNPHKMLLAGIQCCAFLVKDNSGLLKKCYSAKAAYLFQQDKFYDVSYDTGDKSIQCSRRPDAFKFWLMWKALGTTGLEERVNRALALARYLVEEIKKREGFRLLLEPEYANVCFWYIPPSLRKMEDGPEFWQKLHQVAPIIKERMMKKGSMMLGYQPHQGKVNFFRQVVISPQVSREDMDFLLDEIELLAKDL

SEQ ID NO: 17 - protein, 511 aa, Xenopus tropicalis
HDIDKNKQETRPMKRNAVLVDGVVLNGPIIDSSSGEKFVEDVYRILMNELVYKASDINQKVCEWQEPEQLKKLLDLNIKDNGEPHEKLLQLCKNVIKYSVKTSHPRFFNQLYAGMDHYSLAARFITEALNPSVYTYEVSPVFILTEEAILKKMIEFLGWKEGDGIFSPGGSVSNMYAVNLARYKYCPDIKQKGLSSAPRLVMFTSEECHYSMKKAAAFLGIGTENVYFVKTDDRGKMIPEELENQIQRAKKEGAVPFLVSATSGTTVLGAFDPLDDIANICEKHKLWFHVDASWGGSALMSQKYRKRLHGIHRADSVAWNPHKMLMAGIQCCALLVRDNSGLLKRCHSAEATYLFQQDKFYDVQYDTGDKSIQCSRRADAFKFWMMWKALGTTGLEERINRALALTRYLASEIKKRDGFELLWEPEYANTCFWYIPPSFRNMEKGPEYWRKFSNVAPTIKERMMKKGSMMVGYQPHRDKVNFFRHIVISPQVSREDMDFVLDEIERLGRDL

SEQ ID NO: 18 - protein, 511 aa, Daphnia magna
MEVGVCKQEMLQKKKNAVLVDGVILNGPIMDSKAGEKFVEEACKIIMEEVIQKADDVTEKVCEWRAPETLKQILDLEMRDTGETHQKLLQLCRDVIQYSVKTGHPRFFNQLYAGIDYYSLVARFITEALNPSVYTYEVSPVFLLVEEAVIKKMIEFIGWEEGDGIFNPGGSVSNMYAMNLARYKFFPEIKEKGLSGLPRLVLFTSEECHYSMKKAASFLGIGTENVYFIKTDERGKMIPEELEKQVQRARKEGSAPFLVCATAGTTVLGAFDPLDKIADICEKHGLWLHVDASWGGSALISRKHCRLLQGIHRADSVAWNPHKMLLAGIQCCALLVKDNSGLLKKCYSAKAAYLFQQDKFYDVSYDTGDKSIQCSRRPDAFKFWLMWKALGTTGLEQRVNRALALARYLVEEIKKREGFQLLLEPEYANVCFWYIPPSLRKMEDGPEFWQKLHQVAPIIKERMMKKGSMMLGYQPHQDKVNFFRQVVISPHVSREDMDFLLDEIELLAKDL

SEQ ID NO: 19 - protein, 511 aa, Phasianus colchicus
MEASTCKQDVLQRKKNAVLVDGVILNGPITDSKAGEKFVEEACKIIMEEIIQKADDVTEKVCEWRDPETLKKILDLEMRDTGESHQKLLQLCQDVIQYSVKTNHPRFFNQLYAGIDYYSLVARFITEALNPSVYTYEVSPVFLLVEEAVIKKMIEFIGWEEGDGIFNPGGSVSNMYAMNLARYKFCPEIKEKGLSGLPRLVLFTSEECHYSMKKAASFLGIGTENVYFVKTDERGKMIPEELEKQVQRARKEGSAPFLVCATAGTTVLGAFDPLDKIADICEKYDLWLHVDASWGGSALISRKHRRLLRGIQRADSVAWNPHKMLLAGIQCCALLVKDNSGLLKKCYSAKAAYLFQQDKFYDVSYDTGDKSIQCSRRPDAFKFWLMWKALGTTGLEERVNRALALARYLVEEIKKREGFQLLLEPEYANVCFWYIPPSLRKMEDGPEFWQKLHRVAPVVKERMMKKGSMMLGYQPNQGKVNFFRQVVISPQVSREDMDFLLDEIELLAKDL

SEQ ID NO: 20 - protein, 499 aa, Xenopus laevis
MKRNAVLVDGVVLNGPIIDSSSGEKFVEDVYRILMNELVYKASDVNQKVCEWQEPDQLKKLLDLNIKDNGEPHEKLLQLCQNVIKYSVKTSHPRFFNQLYAGMDHYSLAARFITEALNPSVYTYEVSPVFILTEEAILMKMIEFIGWKEGDGIFSPGGSVSNMYAVNLARYKCCPDIKQKGLSSAPRLVMFTSEECHYSMKKAAAFLGIGTENVYFVKTDDRGKMIPEELENQIQRAKKEGAVPFLVSATSGTTVLGAFDPLDNIANICEKHKLWFHVDASWGGSALMSRKYRKCLHGIHRADSVAWNPHKMLMAGIQCCALLVKDNSGLLKRCHSAEATYLFQQDKFYDVQYDTGDKSVQCSRRADAFKFWMMWKALGTTGLEERINRALALTRYLASEIKKRDGFELLWEPEYANTCFWYIPPSFRNMEKGPEYWKNFSKVAPTIKERMMKKGSMMVGYQPHRDKVNFFRHIVISPQVSREDMDFVLDEIERLGRDL